(19) United States
(12) Patent Application Publication
(10) Pub. No.: US 2003/0190648 A1
(43) Pub. Date: Oct. 9, 2003

(54) Method and system for sequence presentation

(75) Inventors: Takehiko Hosoiri, Kawagoe (JP); Takahide Yokoi, Kawagoe (JP); Masako Wagatsuma, Higashimurayama (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/314,321

(22) Filed: Dec. 9, 2002

(30) Foreign Application Priority Data
Apr. 5, 2002 (JP) P2002-103333

(51) Int. Cl. C12Q 1/68; G06F 19/00; G01N 33/48; G01N 33/50

(52) U.S. Cl. 435/6; 702/20

(57) ABSTRACT

Based on a partial sequence of a target gene having an unidentified sequence, a partial sequence corresponding thereto is extracted from a genome sequence by homology search on a database. Exon regions are predicted using plural programs, respectively, and common sequences among the predicted exon regions are extracted. A set of primers is designed based on a combination of a 5′ end sequence and a 3′ end sequence selected from the plurality of common sequences. Amplification using the selected combination of 5′ end and 3′ end sequences as a set of primers results in an amplified gene that may be cloned. A display system for preparing primer sequences and utilizing the prepared primer sequences.

### BACKGROUND OF THE INVENTION

1. Field of the Invention

The present invention relates to a method for predicting exon sequences of a gene based on enormous amounts of genome information of eukaryotic organisms. More specifically, it relates to a display system for preparing primer sequences and utilizing the prepared primer sequences.

2. Description of the Related Art

Information on genome base sequences of human and other organisms has been accumulated. In addition, sequences of human gene regions are translated into proteins which perform vital functions, i.e., sequences of cDNAs are now being clarified by action of a genome project that utilizes technologies for acquiring cDNAs in Japan, and information thereon is being accumulated.

To analyze the function of a gene, it is essential to analyze the function of the protein translated from the gene, as well as to read its sequence information.

Accordingly, it is important to clarify and investigate a novel gene function using a cDNA clone as a starting material, which cDNA clone is obtained based on the sequence information as character strings.

Polymerase chain reactions (PCRs) are widely used to acquire cDNA clones of a gene that has been completely clarified on its sequence information. A cDNA of a coding region of the target gene can relatively easily be obtained by performing PCR using a cDNA prepared from a mRNA and two primers having outside sequences sandwiching the identified coding sequence. However, if sequence information of the target gene has been clarified only partially, sequences sandwiching the target coding sequence are not identified, and a cDNA of the coding region cannot be obtained by the conventional PCRs.

As a possible solution to identify a sequence of an unidentified region based on a partial sequence, rapid amplification of cDNA ends (RACE) has been proposed. In RACE, an oligo DNA having an identified sequence is artificially added to the 5′ end of a cDNA upon or after the preparation of the cDNA from a mRNA, the unidentified region is then amplified by PCR using primers corresponding to an identified partial sequence sandwiching the target unidentified sequence and to a sequence of the artificially added oligo DNA, and a cDNA of the target gene is obtained (FIG. 6).

According to RACE, however, the total length of a cDNA of the target gene cannot be predicted, since the sequence of the target gene is not identified. Accordingly, whether or not a DNA fragment length amplified by PCR is a target region cannot be determined. The unpredictability of the amplification length disables setting of PCR conditions. This is because the temperature and time of PCR directly affect the amplification length and amplification efficiency.

In addition, if the target gene has a very long sequence, all primers and enzymes used in the reaction must be optimized, which further complicates acquisition of the cDNA of the coding region of the target gene having a long sequence.

The RACE requires a reaction process step 45 for adding an oligo DNA artificially to the 5′ end of cDNA as an essential step (FIG. 9). The reaction efficiency in the reaction process step affects the amplification efficiency, since molecules of the resulting cDNA of the target gene including the added oligo DNA serve as a template in amplification. In particular, if the target gene is one in which only a very small amount of a mRNA is expressed, the target gene cannot significantly be obtained by the RACE, since the number of molecules of the oligo DNA before the addition reaction is small. As thus described above, a gene having an unidentified sequence has been obtained by the RACE in many cases but is hardly obtained by this technique as compared with a gene having a completely identified coding sequence.

Sequence information on regions other than the identified partial sequence cannot be obtained according to RACE unless a PCR can be performed between the oligo DNA added to the cDNA and the identified partial sequence in the reaction process step, in which the oligo DNA is artificially added to the 5′ end of the cDNA.

### SUMMARY OF THE INVENTION

The present invention provides a method for presentation of sequences to prepare a common sequence. In this method, initially, a predetermined partial sequence is extracted from a mRNA. A partial sequence of a genome sequence corresponding to the partial sequence of the mRNA is then extracted by homology search on a database. Exon regions within the partial sequence of the genome sequence are predicted using computer programs. In this process, respective exon regions are predicted through the use of respective computer programs. A sequence common to the exon regions predicted through the use of the respective computer programs is prepared as a common sequence.

Such databases include, for example, Gene Bank. Such programs include, for example, GenScan, and FGENESH.

The common sequence thus prepared is a common exon sequence extracted through the use of plural programs and has-high reliability. The 5′ end and the 3′ end of the common sequence can be used as primers for amplification by PCR. According to the conventional techniques, a primer is designed only directed to the oligo DNA artificially added to the end of the cDNA. In contrast, plural sets of primers can be obtained according to the present invention when plural common sequences are obtained, which increases probability of amplification by PCR.

To increase the reliability, the number of the plural programs is preferably large particularly in the case where calculation can be performed on plural types of programs simultaneously. However, if calculation cannot be performed on plural types of programs simultaneously, the upper limit of the number of the programs is preferably set in view of saving time.

After obtaining the plural common sequences, one or more primers in a sense direction and in an antisense direction, respectively, in each common sequence are designed. A sense primer designed in a common sequence and an antisense primer designed in the same or another common sequence are linked to yield one set of primers (hereinafter referred to as “predicted set of primers”). In this manner, plural predicted sets of primers between common sequences are prepared. The use of plural sets of primers increases probability of amplification in the subsequent PCR process step as compared with the use of a single set of primers.

The combinations of the sets of primers are arbitrarily selected based on PCR amplification lengths.

In the above method, common sequences alone are extracted. It is also acceptable that minority sequences are extracted in addition to the common sequences. The term “minority sequence” as used herein means a sequence of an exon region, which exon can be predicted through the use of one program but cannot be predicted through the use of another program. Such minority sequences are preferably extracted when the extracted common sequences cannot yield sufficient information, i.e., when only a few types of common sequences can be extracted and a sufficient number of sets of primers cannot be obtained from combinations of the 5′ ends and the 3′ ends of the common sequences.

Upon selection of a set of primers, the size thereof is preferably set at about 500 to about 1000 bp. An excessively long amplification length deteriorates the amplification efficiency, and in contrast, an excessively short amplification length inhibits amplified products to be identified. The size of the set of primers is not specifically limited to those specified above and is arbitrarily selected depending on amplification conditions such as temperature, time, and type of enzyme used.

After designing the primers, an amplification reaction by PCR is performed using the designed primers with a template cDNA obtained from the mRNA by reverse transcription. The reaction mixture obtained as a result of amplification is subjected to electrophoresis to detect the presence or absence of amplified products. Plural amplified products are purified and are then subjected to sequencing. One sequence is defined as an overlapped region from among sequences of the plural amplification products. In the sequence, a coding sequence is determined, and a primer for amplification of the coding region is designed. The coding region is amplified using a cDNA as a template. The resulting amplified product and a cloning vector or an expression vector are ligated. The reaction mixture is introduced into Escherichia coli, and cDNA clone is obtained on a selection medium.

The present invention also provides a display system having the following configuration.

Initially, a partial sequence is extracted from a mRNA. A partial sequence of a genome sequence corresponding to the partial sequence of the mRNA is then determined by homology search on a genome database.

On a display screen, selecting means for selecting plural programs for predicting exon regions at the 5′ end, the 3′ end or both of the partial sequence is displayed. The selecting means determines programs that predict exons. The programs comprise two or more types of programs, such as GenScan, FGENESH, and Grail.

Respective exon regions predicted through the use of the programs are displayed on the screen on a program basis. The display system further includes a common sequence extraction button that extracts a region common to the respective exon regions predicted through the use of the respective programs. By this configuration, the procedure of extracting a common sequence can be visualized on the display system and can easily be performed.

When plural common sequences are extracted, the screen may display a selecting means for extracting a combination of the 3′ end and the 5′ end of any of the plural common sequences. The selecting means can determine a set of primers and mainly serves to select the length of a sequence to be amplified. The screen may display a box to select the length of the sequence. Alternatively, an operator arbitrarily selects the set of primers by double-clicking on a displayed primer region and the set of primers is selected in a region containing the target common sequence. On the display screen, primers not used may be omitted by filtering.

The display system preferably further includes a sequence display means for displaying the sequence of the selected set of primers. Subsequent to the above procedures, one prepares the target primers and then performs PCR and other operations. By displaying the sequence of the set of primers, one can prepare the primers with reference to the sequence with very good workability.

The display system preferably further includes a sequence display means for displaying the sequence of a region to be sandwiched between the selected set of primers. The sequence of the set of primers can be output as data to be compared with actually determined data.

In addition, the display system may preferably include a minority sequence extraction button for extracting an exon region that is predicted through the use of a predetermined program but is not predicted through the use of another program. When the use of a selected set of primers obtained by extracting the common sequences alone does not contribute to amplification, the minority sequence should preferably be extracted.

By extracting common sequences, reliability on exon sequence information is improved, and a large number of candidates for sets of primers can be obtained. Accordingly, a cDNA clone of a total coding region can be obtained from a partial sequence of a gene having an unidentified sequence by PCR with an almost equivalent efficiency to the acquisition of a cDNA clone of a gene having an identified sequence.

Other and further objects, features and advantages of the invention will appear more fully from the following description.

### BRIEF DESCRIPTION OF THE DRAWINGS

This invention is to be described specifically for preferred embodiments with reference to the drawings. Throughout the drawings for explaining the preferred embodiments, those having identical functions carry the same reference numerals, for which duplicate explanations have been omitted, wherein:

FIG. 1 is a schematic diagram showing a cloning process using a database according to the present invention;

FIG. 2 shows process steps to design of a predicted set of primers from partial sequence information;

FIG. 3 is a flow chart of process steps for designing the predicted primers;

FIG. 4 shows process steps of selecting common sequences and of selecting sets of primers designed in the common sequences;

FIG. 5 illustrates process steps of performing a PCR using the designed sets of primers, of determining a coding region of a target gene, and of cloning based on information thereof;

FIG. 6 illustrates a display screen image of Basic Local Alignment Search Tool (BLAST) search for a partial sequence;

FIG. 7 schematically illustrates information of position of the predicted exon sequence as a box object in a window showing the prediction;

FIG. 8 shows process steps of identifying a genome region of a partial sequence, of predicting exon sequences using prediction programs, and of identifying common sequences among the exon sequences predicted through the use of plural prediction programs;

FIG. 9 shows process steps of RACE as a conventional technique; and

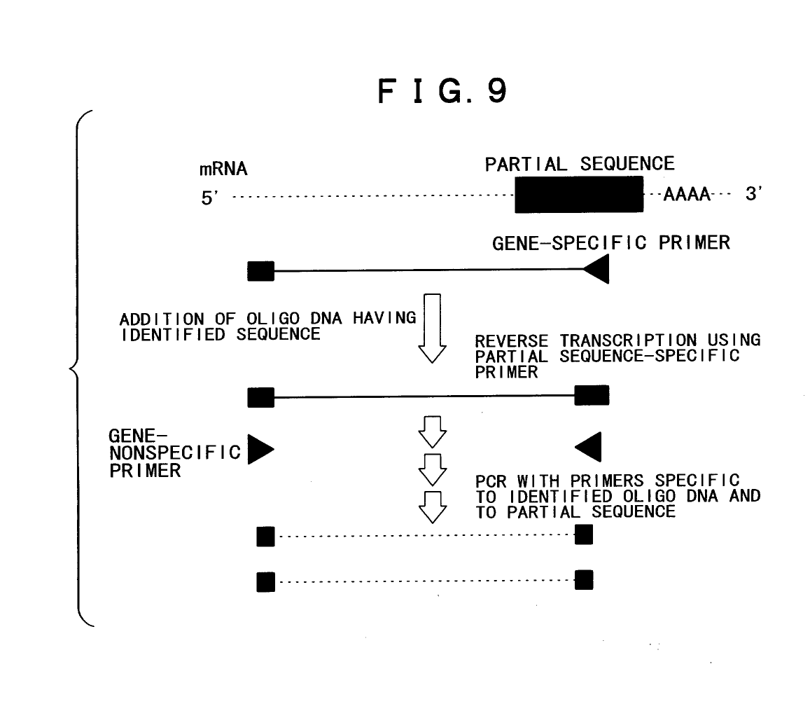

FIG. 10 shows the result of BLAST search of a partial sequence in an example of the present invention.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, other elements that may be well known. Those of ordinary skill in the art will recognize that other elements are desirable and/or required in order to implement the present invention. However, because such elements are well known in the art, and because they do not facilitate a better understanding of the present invention, a discussion of such elements is not provided herein. The detailed description the preferred embodiments of the present invention will be provided herein below with reference to the attached drawings.

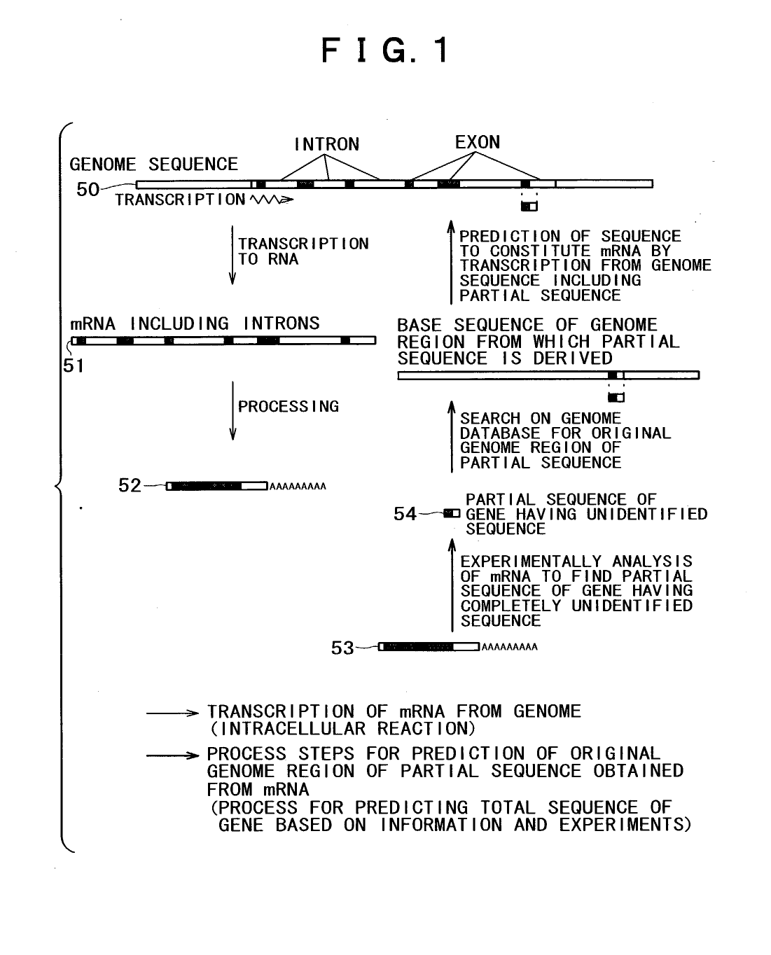

FIG. 1 schematically illustrates cloning process steps using a database according to the present invention. An organism synthesizes (transcripts) a mRNA from a genome sequence 50. The mRNA will serve as a template in protein synthesis (translation). The genome sequence 50 includes “exon” regions carrying genetic information necessary for protein synthesis and “intron” regions carrying no such information. The two types of regions are arrayed alternately. Initially, a mRNA 51 including the both regions is transcribed. The mRNA 51 then undergoes processing to remove introns to thereby form a mRNA 52 carrying continuous sequence information comprising the exons alone. The mRNA 52 containing no introns serves as a template in protein synthesis.

A partial sequence 54 of a gene having unidentified sequence is experimentally obtained from part of a mRNA 53 containing no introns. The mRNA 53 having the partial sequence 54 has been synthesized (transcribed) from the genome 50, and it should be understood that the genome 50 carries a region having the partial sequence 54. By searching an accumulated database of genome sequences for a region corresponding to the partial sequence 54, a genome region at which the transcribed gene is present can be identified. However, the identified genome region carries a sequence containing exons and introns arrayed alternately.

Accordingly, the present invention provides a method for cloning a gene having an unidentified sequence. In this method, a sequence comprising exons alone is extracted from the genome sequence using programs for predicting exons and introns to thereby predict sequence information of a mRNA containing no introns. Primers are then designed based on the sequence information and are subjected to amplification to thereby clone the gene having an unidentified sequence.

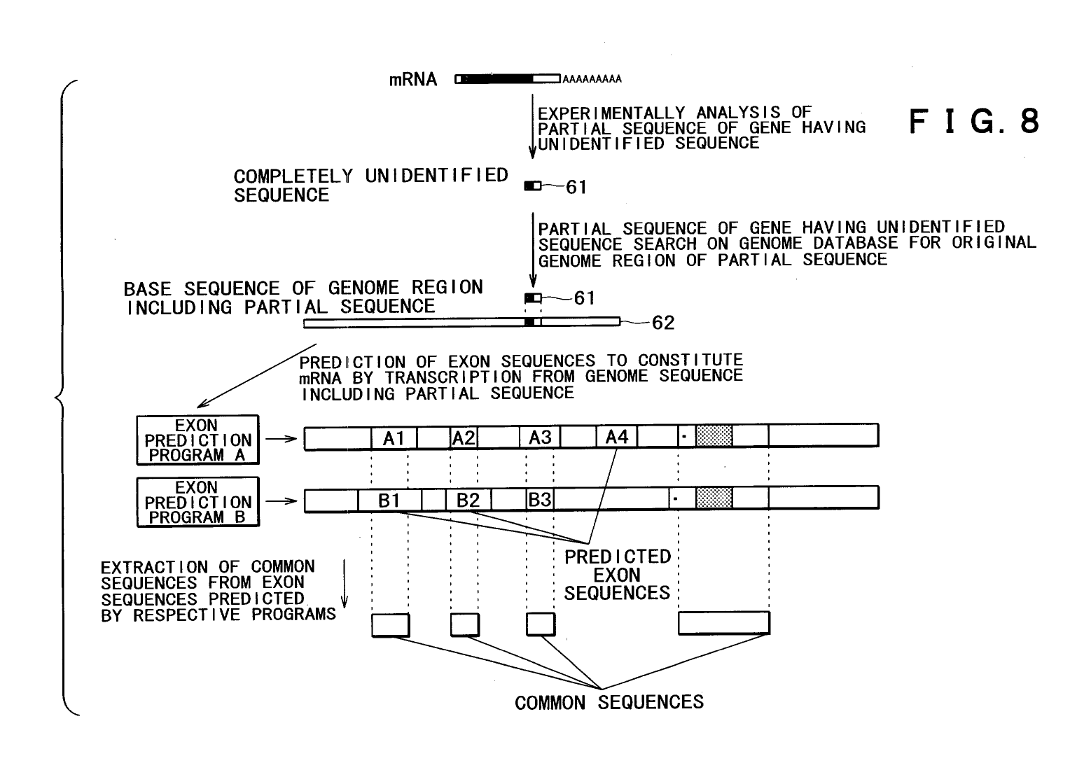

FIG. 8 shows process steps according to the present invention of identifying a genome region of a partial sequence, of predicting exon sequences using prediction programs, and of specifying common sequences among the exon sequences predicted through the use of plural prediction programs. A genomic database is searched for partial sequence information 61 of the gene having an unidentified sequence, and a genome sequence 62 containing the partial sequence is extracted. Exon sequences are predicted (hereinafter referred to as “predicted exon sequences”) based on the genome sequence 62 using plural exon prediction programs. The predicted exon sequences determined through the use of the respective programs are compared with one another to thereby extract common sequences among the predicted exon sequences (hereinafter referred to as “common sequences”).

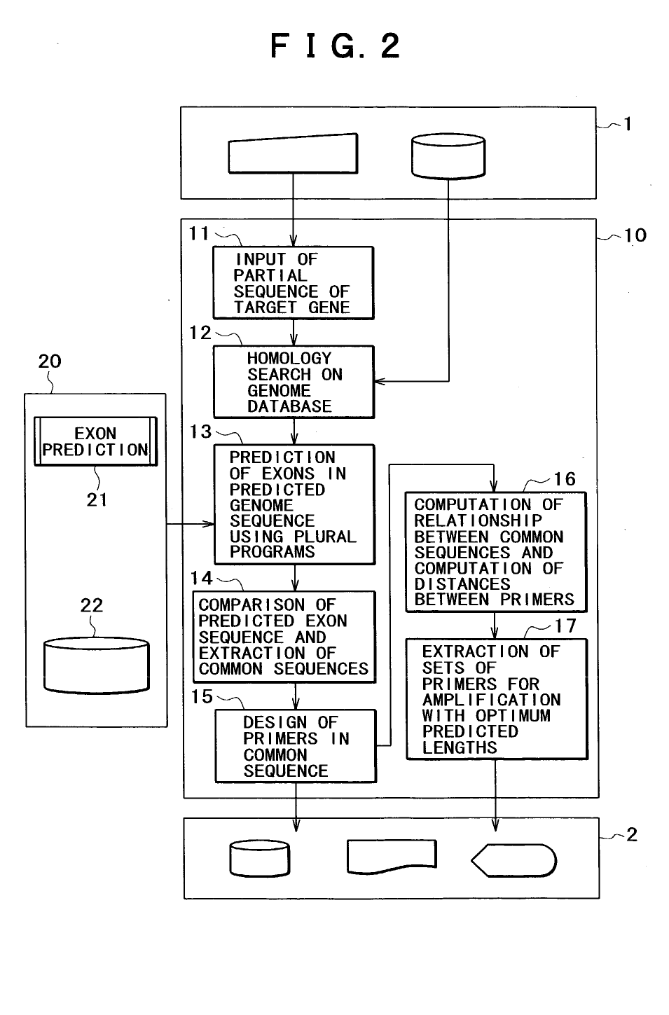

FIG. 2 shows process steps according to the present invention for the design of a predicted set of primers based on the partial sequence information.

FIG. 2 illustrates an input device 1, an output device 2, and processing units (CPUs and memories) 10 and 20 for primer design. The processing unit 10 comprises a partial sequence input unit 11 for entering the partial sequence of the target gene, a homology search unit 12 for searching the genomic database, an exon prediction unit 13 for predicting exons in the predicted genome sequence, a comparison and extraction unit 14 for comparing the predicted exon sequences and extracting the common sequences, a designing unit 15 for designing primers in the common sequences, a computing unit 16 for calculating relative relations between the common sequences and distances between individual primers, and a primer set extraction unit for extracting sets of primers for the amplification with optimum predicted lengths. The processing unit 20 comprises plural programs 21 and databases 22 on sequences. The programs 21 have algorithms for prediction of exons and are used in the processing in the exon prediction unit 13 in the processing unit 10. The databases 22 are used for the prediction of the exons.

The process steps of identifying the position of the target gene on the genome based on the partial sequence information, of extracting the predicted exon sequences from the genome sequence, and of designing primers are illustrated in detail below.

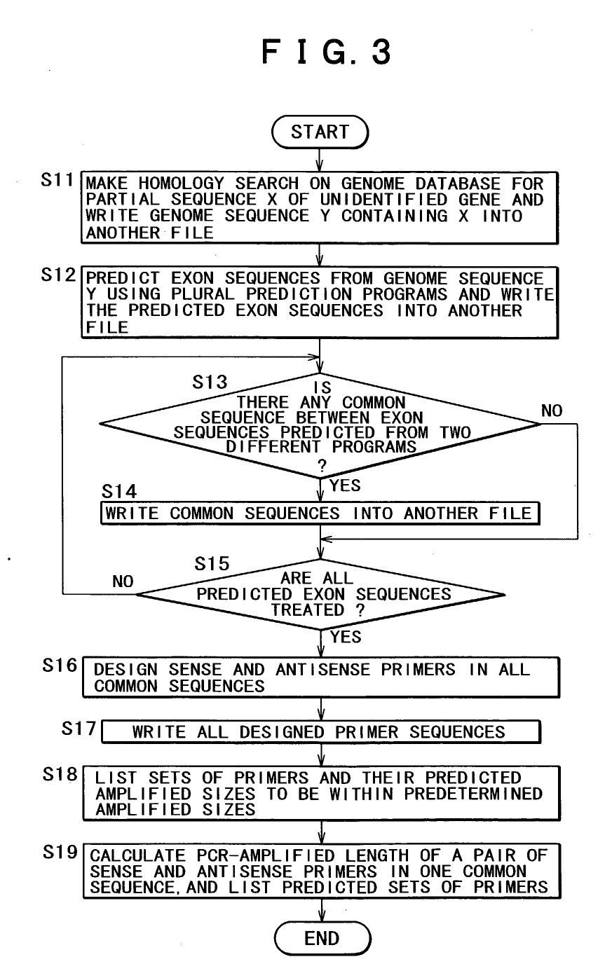

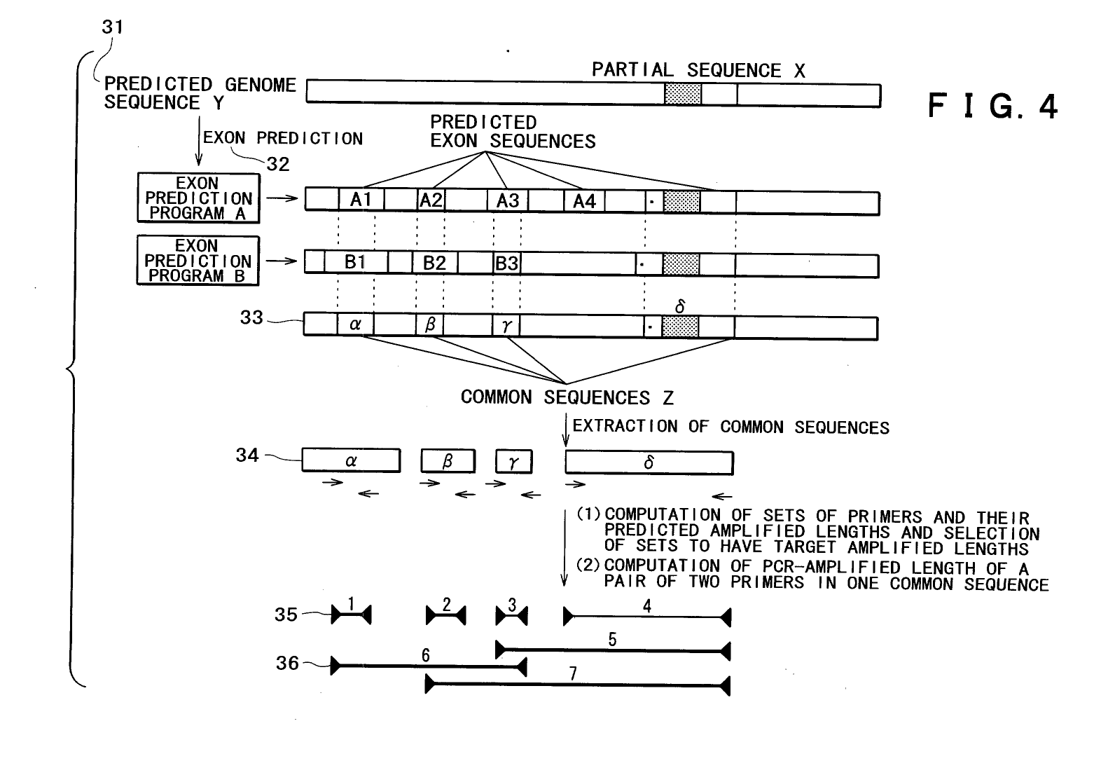

FIG. 3 is a flow chart of the design process steps for the predicted primers, and FIG. 4 shows process steps of selecting common sequences and of selecting sets of primers designed in the common sequences.

With reference to FIGS. 3 and 4, a genomic database is homology-searched for a partial sequence X, a predicted genome sequence Y containing the partial sequence X is extracted, and the sequence datum thereof is written into another file (Step S11 in FIG. 3 and Step 31 in FIG. 4). The predicted genome sequence Y is a continuous genome sequence straddling the partial sequence X and is extracted from the sequences on the database.

The predicted genome sequence Y is subjected to two exon prediction programs and thereby the predicted exon sequences are extracted from the predicted genome sequence Y and are written into other files (Step S12 in FIG. 3 and Step 32 in FIG. 4). The two exon prediction programs used herein are GenScan [Burge, C. and Karlin, S. (1997) “Prediction of complete gene structures in human genomic DNA” J. Mol. Biol. 268: 78-94] and FGENESH [Salamov A. A., Solovyev V. V., (1999), unpublished data, refer to Kulp, D., Haussler, D., Reese, M. G., and Eeckman, F. H. (1996), Proc. Conf. on Intelligent Systems in Molecular Biology, 134-142]. By subjecting the predicted genome sequence Y to plural exon prediction programs, extraction of false positive sequences and omission of false negative sequences can be prevented. These problems occur when the sequences are extracted by using only one prediction algorithm of one program.

Next, the respective predicted exon sequences written into the files extracted through the use of the plural programs are compared with one another, and common sequences Z are written into another file (Steps S13 to S15 in FIG. 3). In this procedure, the seriality (order) of the respective common sequences Z keeps the seriality in the genome sequence Y (Step 33 in FIG. 4).

Subsequently, primers are designed in a sense direction and in an antisense direction in all the common sequences Z, and the sequences of the designed primers are written into a file (Steps S16 and S17 in FIG. 3 and Step 34 in FIG. 4).

Approximate predicted sizes of PCR amplification products of all the combinations of any designed primer (in a sense direction) of any common sequence and another designed primer (in an antisense direction) of any other common sequence are calculated based on the positional relations among the common sequences Z, and sets of primers to have predetermined amplified sizes and their predicted amplified sizes are listed (Step S18 in FIG. 3 and Step 36 in FIG. 4).

In addition, an approximate predicted size of a PCR amplification product using a sense primer and an antisense primer in any one common sequence is calculated, this procedure is repeated, and the resulting sets of primers and their predicted amplified sizes are listed (Step S19 in FIG. 3 and Step 35 in FIG. 4).

cDNA of a coding region of the target gene is then cloned using primers belonging to the sets of primers thus designed and output. The process steps for this cloning procedure are illustrated in detail below.

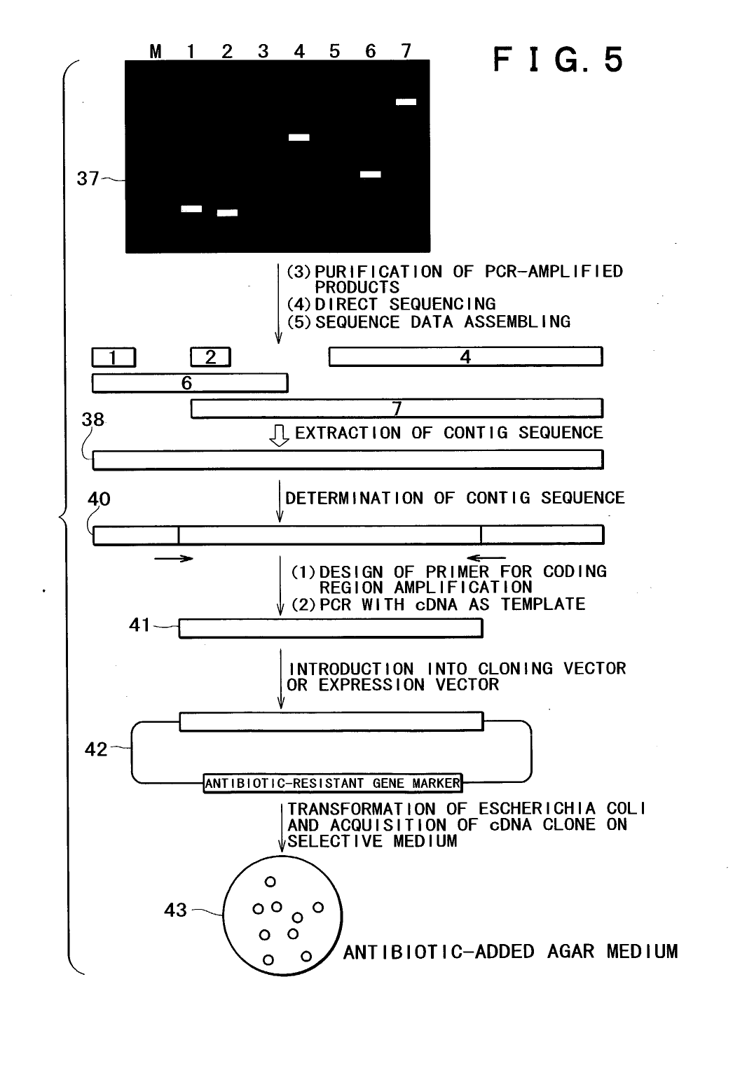

FIG. 5 illustrates process steps of performing a PCR using the designed sets of primers, of specifying a coding region of a target gene, and of cloning based on information thereof.

Initially, PCRs are performed using the sets of primers having the designed sequences, and the presence or absence of PCR amplification products are detected by electrophoresis (Step 37 in FIG. 5).

The respective amplification products are purified and are then subjected to cycle sequencing to thereby yield sequencing samples, and the sequencing samples are subjected to a sequencer to identify the sequences.

The resulting sequence data are linked at the common sequence regions to thereby yield one serial sequence (Step 38 in FIG. 5). In addition, a coding sequence in the serial sequence is determined (Step 40 in FIG. 5).

Primers corresponding to sequences outside the coding region are designed, and the designed primers are subjected to a PCR using a cDNA as a template to thereby amplify the coding region (Steps 40 and 41 in FIG. 5).

The resulting amplified product is ligated with a cloning vector or an expression vector and is introduced into Escherichia coli (Step 42 in FIG. 5). The treated Escherichia coli is then cultured on an antibiotic-added agar medium. By action of an antibiotic resistance marker coded on the vector, Escherichia coli carrying the vector can selectively grow on the antibiotic-added agar medium, and thus the coding region of the target gene cloned on the vector is cloned (Step 43 in FIG. 5).

The present invention will be illustrated in further detail with reference to several examples below, which are not intended to limit the scope of the invention.

### EXAMPLE 1

### Method for Presenting Predicted Sequences

1. Determination of Genome Sequence and Extraction of Predicted Sequences from Partial Sequence of Unidentified Human Gene.

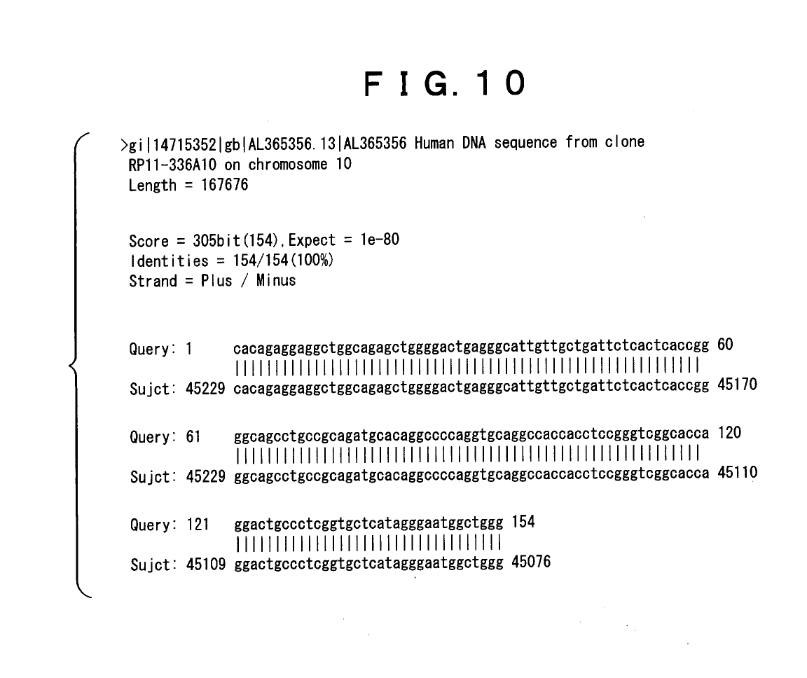

A partial sequence (SEQ ID NO: 1) experimentally obtained from a human mRNA was subjected to BLAST search [Altschul, S. F., Gish, W., Miller, W., Myesrs, E. W., and Lipman, D. J. (1990) “Basic local alignment search tool” J. Mol. Biol. 215:403-410] on GenBank (http://www.ncbi.nlm.nih.gov/Genbank/index.html). As a result, the partial sequence coincided with part of a genome sequence (FIG. 10). The partial sequence in question is a sequence corresponding to 45076-45230 of AL365356 and is represented by SEQ ID NO: 2.

The obtained genome sequence (SEQ ID NO: 2) was then subjected to two exon prediction programs to thereby extract exon sequences predicted from the genome sequence. The two exon prediction programs used herein are GenScan [Burge, C. and Karlin, S. (1997) “Prediction of complete gene structures in human genomic DNA” J. Mol. Biol. 268: 78-94] and FGENESH [Salamov A. A., Solovyev V. V., (1999), unpublished data, refer to Kulp, D., Haussler, D. , Reese, M. G. , and Eeckman, F. H. (1996), Proc. Conf. on Intelligent Systems in Molecular Biology, 134-142].

The predicted exon sequences are as follows.

[Exon Sequences Predicted by GENSCAN]

Exon A1 (SEQ ID NO: 3)

Exon A2 (SEQ ID NO: 4)

Exon A3 (SEQ ID NO: 5)

Exon A4 (SEQ ID NO: 6)

Exon A5 (SEQ ID NO: 7)

Exon A6 (SEQ ID NO: 8)

Exon A7 (SEQ ID NO: 9)

Exon A8 (SEQ ID NO: 10)

Exon A9 (SEQ ID NO: 11)

Exon A10 (SEQ ID NO: 12)

Exon A11 (SEQ ID NO: 13)

Exon A12 (SEQ ID NO: 14)

Exon A13 (SEQ ID NO: 15)

Exon A14 (SEQ ID NO: 16)

Exon A15 (SEQ ID NO: 17)

Exon A16 (SEQ ID NO: 18)

[Exon Sequences Predicted by FGENESH]

Exon B1 (SEQ ID NO: 19)

Exon B2 (SEQ ID NO: 20)

Exon B3 (SEQ ID NO: 21)

Exon B4 (SEQ ID NO: 22)

Exon B5 (SEQ ID NO: 23)

Exon B6 (SEQ ID NO: 24)

Exon B7 (SEQ ID NO: 25)

Exon B8 (SEQ ID NO: 26)

Exon B9 (SEQ ID NO: 27)

Exon B10 (SEQ ID NO: 28)

2. Extraction of Common Sequences of Predicted Exon Sequences

The exon sequences extracted from the genome sequence were compared using individual prediction programs to find that combinations of Exon A1 with Exon B1, Exon A6 with Exon B3, Exon A8 with Exon B4, Exon A9 with Exon B5, Exon A11 with Exon B6, Exon A12 with Exon B7, Exon A13 with Exon B8, Exon A15 with Exon B9, and Exon A16 with Exon B10 have common sequences, respectively. These common sequences were then extracted.

Common sequence C1 between Exon A1 and Exon B1 (SEQ ID NO: 29)

Common sequence C2 between Exon A6 and Exon B3 (SEQ ID NO: 30)

Common sequence C3 between Exon A8 and Exon B4 (SEQ ID NO: 31)

Common sequence C4 between Exon A9 and Exon B5 (SEQ ID NO: 32)

Common sequence C5 between Exon A11 and Exon B6 (SEQ ID NO: 33)

Common sequence C6 between Exon A12 and Exon B7 (SEQ ID NO: 34)

Common sequence C7 between Exon A13 and Exon B8 (SEQ ID NO: 35)

Common sequence C8 between Exon A15 and Exon B9 (SEQ ID NO: 36)

Common sequence C9 between Exon A16 and Exon B10 (SEQ ID NO: 37)

### EXAMPLE 2

### Primer Design

1. Primer Design Based on Common Sequences

Sense primers and antisense primers of the common sequences were designed using a primer designing software Oligo (available from Molecular Biology Insights, Inc.). In this procedure, a sense primer and an antisense primer were designed upstream and downstream of a target common sequence in the opposite directions to each other.

The sense primers F and antisense primers R designed on the common sequences are as follows.

a. Designed from Common Sequence C1 (SEQ ID NO: 29)

| | |
|---|---|
| Primer C1-F: | |
| 5′-GAAACAGTGATTATGAACACCG-3′ | (SEQ ID NO:38) |
| Primer C1-R: | |
| 5′-GCGACCGAGCCGGGAGT-3′ | (SEQ ID NO:39) |

b. Designed from Common Sequence C2 (SEQ ID NO: 30)

| | |
|---|---|
| Primer C2-F: | |
| 5′-GGAGCGGACCCCTGTGC-3′ | (SEQ ID NO:40) |
| Primer C2-R: | |
| 5′-CAGCCGCCAGCAGCAG-3′ | (SEQ ID NO:41) |

C. Designed from Common Sequence C3 (SEQ ID NO: 31)

| | |
|---|---|
| Primer C3-F: | |
| 5′-CGCAACATCGACGGCAG-3′ | (SEQ ID NO: 42) |
| Primer C3-R: | |
| 5′-CAGGGGGGACGCTGTGTA-3′ | (SEQ ID NO: 43) |

d. Designed from Common Sequence C4 (SEQ ID NO: 32)

| | |
|---|---|
| Primer C4-F: | |
| 5′-TGTGTGAGCCTTCTTATTGACG-3′ | (SEQ ID NO:44) |
| Primer C4-R: | |
| 5′-GCAGCACTTTGACACAGTCCAG-3′ | (SEQ ID NO:45) |

e. Designed from Common Sequence C5 (SEQ ID NO: 33)

| | |
|---|---|
| Primer C5-F: | |
| 5′-GAGACTGCCCTTCACCACG-3′ | (SEQ ID NO:46) |
| Primer C5-R: | |
| 5′-AGCACTTGGCGGGAGC-3′ | (SEQ ID NO:47) |

f. Designed from Common Sequence C6 (SEQ ID NO: 34)

| | | |
|---|---|---|
| Primer C6-F: | 5′-CCGGACCGTGGCTGC-3′ | (SEQ ID NO:48) |
| Primer C6-R: | 5′-GGGCAATGCTGGGCAC-3′ | (SEQ ID NO:49) |

g. Designed from Common Sequence C7 (SEQ ID NO: 35)

| | |
|---|---|
| Primer C7-F: | |
| 5′-TACAGAACCTACCCTCTCAATG-3′ | (SEQ ID NO:50) |
| Primer C7-R: | |
| 5′-CTGCACCTGGGGCCTGT-3′ | (SEQ ID NO:51) |

h. Designed from Common Sequence C8 (SEQ ID NO: 36)

| | |
|---|---|
| Primer C8-F: | |
| 5′-TGATGCCAACTTCAGCACC-3′ | (SEQ ID NO:52) |
| Primer C8-R: | |
| 5′-CCCGTGGACAGCGTCTG-3′ | (SEQ ID NO:53) |

i. Designed from Common Sequence C9 (SEQ ID NO: 37)

| | |
|---|---|
| Primer C9-F: | |
| 5′-GTTTCTTCTAGGCAGTTGAGTTC-3′ | (SEQ ID NO:54) |
| Primer C9-R: | |
| 5′-CCTTCAAGCCAAAATCACTGAG-3′ | (SEQ ID NO:55) |

2. Selection of Sets of Primers for Use in PCR

On the assumption that the common sequences are sequences of the gene to be cloned, predicted PCR amplification lengths were calculated on all the combinations of the designed sense primers and antisense primers, and 18 sets of primers which were predicted to have amplification lengths of from 451 bp to 1057 bp were selected. The combinations are as follows.

Primer C1-F and Primer C1-R (predicted amplification length: 623 bp)

Primer C1-F and Primer C2-R (predicted amplification length: 897 bp)

Primer C1-F and Primer C3-R (predicted amplification length: 1037 bp)

Primer C2-F and Primer C4-R (predicted amplification length: 451 bp)

Primer C2-F and Primer C5-R (predicted amplification length: 634 bp)

Primer C2-F and Primer C6-R (predicted amplification length: 691 bp)

Primer C2-F and Primer C7-R (predicted amplification length: 921 bp)

Primer C2-F and Primer C8-R (predicted amplification length: 1057 bp)

Primer C3-F and Primer C6-R (predicted amplification length: 504 bp)

Primer C3 F and Primer C7-R (predicted amplification length: 734 bp)

Primer C3-F and Primer C8-R (predicted amplification length: 870 bp)

Primer C4-F and Primer C7-R (predicted amplification length: 584 bp)

Primer C4-F and Primer C8-R (predicted amplification length: 720 bp)

Primer C4-F and Primer C9-R (predicted amplification length: 1002 bp)

Primer C5-F and Primer C8-R (predicted amplification length: 570 bp)

Primer C5-F and Primer C9-R (predicted amplification length: 852 bp)

Primer C6-F and Primer C9-R (predicted amplification length: 685 bp)

Primer C7-F and Primer C9-R (predicted amplification length: 613 bp)

### EXAMPLE 3

### Display System

1. Schematic Display of BLAST Search and Exon Prediction:

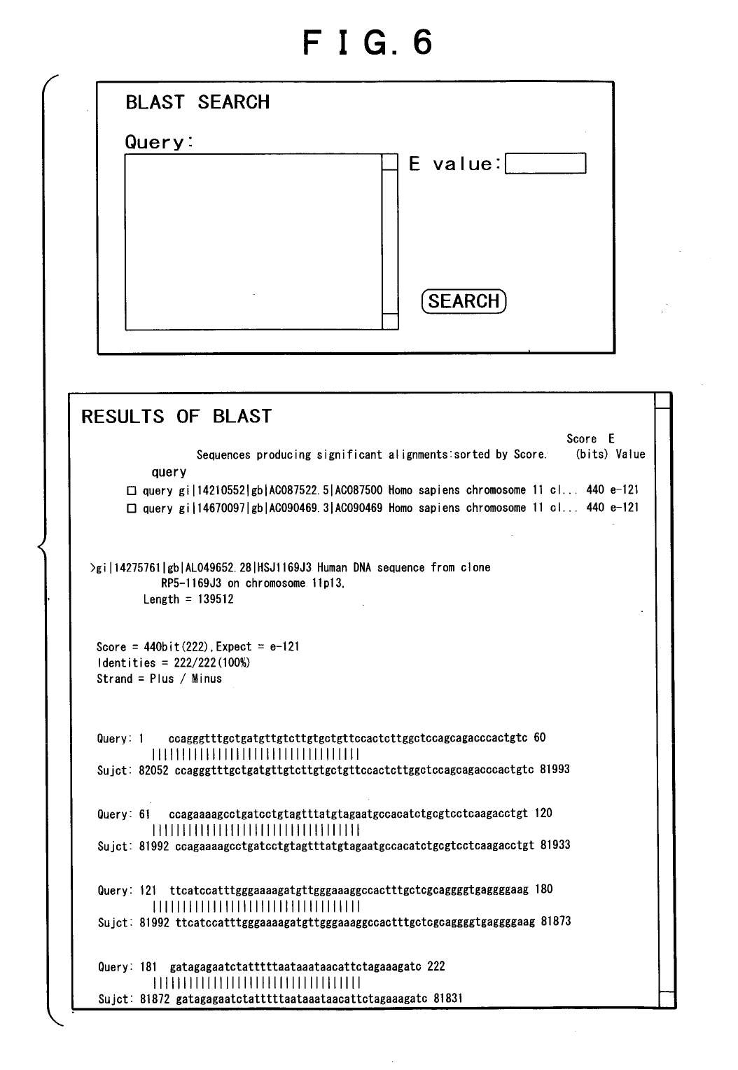

(1) The partial sequence in question is input into Query window of a BLAST search screen (FIG. 6), and the lower limit of E value is input into an E value window to narrow the scope of the search.

(2) A click on a search button triggers a BLAST search, and a list of the results and regions homologous to the partial sequence are shown in a BLAST search output window.

(3) With a click on a check box on the left side of the BLAST search output based on information on the homologous regions to the partial sequence, a genome sequence to be adapted is selected.

(4) A click on a selection button makes a region on the adapted genome sequence including the partial sequence automatically undergo the exon prediction programs.

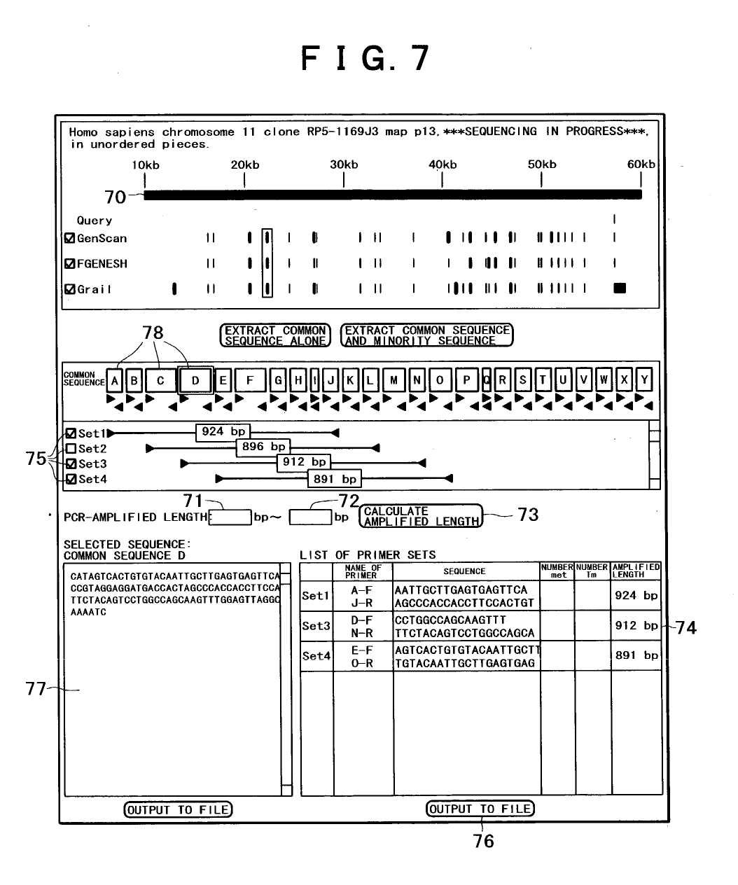

(5) Information on origin of the adapted genome sequence, the position of the partial sequence with respect to the genome sequence, and position information of exon sequences predicted through the use of the respective programs are schematically shown as box objects on a prediction output window 70. (FIG. 7).

(6) On the prediction output window 70, outputs of the plural prediction programs can be compared concurrently. By unmarking a checkbox on the left side of the name of each prediction program, the result of the prediction program showing an extremely different disappears from the screen.

(7) With a click on “Extract Common Sequence Alone” button, common sequences common to the exon sequences predicted through the use of the respective programs are schematically shown as box objects on a common sequence window.

(8) At the same time, sense primers and antisense primers are automatically designed on the respective common sequences and are schematically shown as arrow objects on the common sequence window.

(9) A range of PCR amplification lengths is input in number input windows 71, 72 with a click on a calculation button. By this procedure, sets of primers which are predicted to have amplification lengths within the calculated range are selected from combinations of the sense-primers and antisense primers designed in the step (8). The selected sets of primers and their predicted amplification lengths are schematically shown on a primer set window 74.

(10) By marking one of the checkboxes 75 on the left side of each of the sets of primers, desired sets of primers can be selected. The names, sequences, numbers of bases, melting temperatures (Tms), and predicted amplification lengths of the selected sets of primers are listed.

(11) After the selection of sets of primers and display of the primer list, the “OUTPUT TO FILE” button 76 is selected to store the primer list as a file.

(12) With a click on any of the schematic displays (the genome sequence, partial sequence, exon sequences predicted through the use of the programs, box objects of the common sequences, and arrow objects of the primer sequences), a sequence corresponding to the clicked object 78 is displayed on a sequence window 77. With a click on an object of a common sequence, a predicted exon sequence from which the common sequence is derived is shown in box 77. In contrast, with a click on an object of a predicted exon sequence, a common sequence derived from the clicked predicted exon sequence is shown in box 77.

(13) In this procedure, a minority sequence is defined as a sequence which is not predicted by at least one program, among the exon sequences predicted through the use of the plural programs. Primers may be designed also on such a minority sequence.

(14) Primers are synthetically prepared based on the resulting sets of primers and are subjected to PCRs.

### EXAMPLE 4

### Determination of Coding Sequence

1. PCRs Using Sets of Primers

1-1. Reverse Transcription of mRNA (cDNA Synthesis)

A total of 250 ng of brain mRNA (available from Clontech) was added to 10 pmol of an oligo d(T) primer (SEQ ID NO: 56), sterile water was added to the resulting mixture to a total volume of 4.9 ml, the mixture was left stand at 70° C. for 10 minutes and was then cooled on ice. To the resulting mixture, 2.4 ml of 25 mM MgCl₂, 1.0 ml of 10 mM dNTPs, 0.2 ml of 0.1 M DTT, and 1.0 ml of 10 times reverse transcription (10×RT) buffer [200 mM Tris-HCl (pH 8.4), and 500 mM KCl] were added to a total volume of 9.5 ml. The mixture was warmed at 42° C. for 5 minutes and was then subjected to a reverse transcription reaction at 42° C. for 60 minutes with 25 units (0.5 ml) of SuperScript II reverse transcriptase (available from GIBCO BRL). The reaction mixture was heated at 70° C. for 15 minutes to deactivate SuperScript II to thereby terminate the reaction. RNAs in the reaction mixture were decomposed with 0.5 ml of RNAse by warming at 37° C. for 20 minutes.

1-2. PCR Using cDNA as Template

On each of the sets of primers, 10 pmol each of a sense primer and an antisense primer, 3.2 ml of 2.5 mM dNTPs, 2 ml of 10×buffer [final concentration: 10 mM Tris-HCl (pH 8.3), 50 mM KCl, and 1.5 mM MgCl₂], and 0.5 unit of Taq DNA polymerase (available from Perkin Elmer, Inc.) were mixed, sterile water was added thereto to a total volume of 20 ml, followed by PCR using 0.4 ml of the cDNA prepared in the step 1-1. In the PCR, a first denaturation was performed at 94° C. for 5 minutes. Subsequently, an amplification reaction was performed by repeating, 35 times, a cycle of denaturation at 94° C. for 30 seconds, annealing at 55° C. to 60° C. for 30 seconds, and elongation at 72° C. for 1 minute. At last, elongation was performed at 72° C. for 5 minutes.

A total of 2 ml of PCR products was subjected to electrophoresis on 1% agarose gel to detect amplified bands.

Amplified products in the following sets of primers were detected.

Primer C2-F and Primer C4-R

Primer C2-F and Primer C5-R

Primer C2-F and Primer C6-R

Primer C2-F and Primer C7-R

Primer C3-F and Primer C6 R

Primer C3-F and Primer C7-R

Primer C4-F and Primer C7-R

2. Sequencing of PCR Amplified Products

2-1. Purification of PCR Amplified Products

The total amount of the seven PCR amplified products was subjected to electrophoresis on 1% agarose gel, target amplified products were cut from the gel and were purified using QIAquick Gel Extraction Kit (available from QIAGEN) in accordance with a protocol in a manual attached to the kit.

2-2. Sequencing of PCR Amplified Products

Each of the PCR amplified products was subjected to cycle sequencing and purification using 100 ng of the purified PCR amplified product as a template and 3.2 pmol of the sense primer used in the PCR in question with ABI PRISM BigDye (TM) Terminators v2.0 Ready Reaction Cycle Sequencing Kit (available from Applied Biosystems) in accordance with a protocol in a manual attached to the kit.

The results of sequencing of the amplified products of the respective sets of primers are as follows.

Base sequence of the amplified product of Primer C2-F and Primer C4-R: (SEQ ID NO: 57)

Base sequence of the amplified product of Primer C2-F and Primer C5-R: (SEQ ID NO: 58)

Base sequence of the amplified product of Primer C2-F and Primer C6-R: (SEQ ID NO: 59)

Base sequence of the amplified product of Primer C2-F and Primer C7-R: (SEQ ID NO: 60)

Base sequence of the amplified product of Primer C3-F and Primer C6-R: (SEQ ID NO: 61)

Base sequence of the amplified product of Primer C3-F and Primer C7-R: (SEQ ID NO: 62)

Base sequence of the amplified product of Primer C4-F and Primer C7-R: (SEQ ID NO: 63)

2-3. Assembling of Sequence Data and Determination of Coding Sequence

The sequences (SEQ ID NO: 57 to 63) of the amplified products of the respective sets of primers and the partial sequence (SEQ ID NO: 1) were subjected to assembling using an assembly software SEQUENCHER (TM) (available from Gene Codes Corporation) and thereby yielded a cDNA sequence (SEQ ID NO: 64) of 1251 bp. From the cDNA, a coding region (SEQ ID NO: 65) of 462 bp was identified.

### EXAMPLE 5

### Cloning Method

1. PCR and Sequencing of Coding Region

1-1. Design of Primers

Primers for the amplification of the coding region were designed based on the determined coding sequence (SEQ ID NO: 65).

1-2. PCR Using cDNA

PCR was performed in the following manner. Using the cDNA prepared in the step 1-1 in Example 3 as a template, each 10 pmol of the primers prepared in the step 1-1 in Example 5, 3.2 ml of 2.5 mM dNTPs, 2 ml of 10×buffer (final concentration: 10 mM Tris-HCl (pH 8.3), 50 mM KCl, and 1.5 mM MgCl₂), and 0.5 unit of Taq DNA polymerase (available from Perkin Elmer) were mixed, sterile water was added thereto to a total volume of 20 ml. In the PCR, a first denaturation was performed at 94° C. for 5 minutes. Subsequently, an amplification reaction was performed by repeating, 35 times, a cycle of denaturation at 94° C. for 30 seconds, annealing at 55° C. to 60° C. for 30 seconds, and elongation at 72° C. for 1 minute. At last, elongation was performed at 72° C. for 5 minutes to thereby yield target PCR amplified products. A total of 2 ml of the PCR products was subjected to electrophoresis on 1% agarose gel to thereby detect the amplified products.

2. Sequencing of PCR Amplified Products

2-1. Purification of PCR Amplified Products

A total amount of the PCR amplified products was subjected to electrophoresis on 1% agarose gel, target amplified products were cut from the gel and were purified using QIAquick Gel Extraction Kit (available from QIAGEN) in accordance with a protocol in a manual attached to the kit.

2-2. Sequencing of PCR Amplified Products

Each of the PCR amplified products was subjected to cycle sequencing and purification using 100 ng of the purified PCR amplified product as a template and 3.2 pmol of the sense primer used in the PCR in question with ABI PRISM BigDye (TM) Terminators v2.0 Ready Reaction Cycle Sequencing Kit (available from Applied Biosystems) in accordance with a protocol in a manual attached to the kit.

2-3. Assembling of Sequence Data

3. Subcloning of PCR Amplified Product

The PCR product amplified by the step 1 above was purified and was then subjected to ligation with pGEM-T vector. Part of the reaction mixture was mixed with 40 ml of competent cells (XL1-Blue), was heated at 42° C. and was inoculated to a Luria-Bertani (LB) agar medium containing 50 mg/ml ampicillin, followed by incubation at 37° C. for 17 hours to allow colonies to grow.

### EXAMPLE 6

### Results Presentation

Part of the colonies grown on the agar medium was cultured in 1 ml of a LB liquid medium at 37° C. for 17 hours under shaking. The resulting culture mixture was centrifuged with a centrifuge (available from Hitachi High-Technologies Corporation under the trade name of HIMAC CF15R) at 3000 rpm for 5 minutes to thereby remove the supernatant LB medium. A plasmid DNA was extracted according to the alkali-sodium dodecyl sulfate (alkali-SDS) method. Using the extracted plasmid DNA as a template, sequencing was performed with the primers designed within the coding region. The result of sequencing and the plasmid DNA were submitted.

While the present invention has been described with reference to what are presently considered to be the preferred embodiments, it is to be understood that the invention is not limited to the disclosed embodiments. On the contrary, the invention is intended to cover various modifications and equivalent arrangements included within the sprit and scope of the appended claims. The scope of the following claims is to be accorded the broadest interpretation so as to encompass all such modifications and equivalent structures and functions.

The foregoing invention has been described in terms of preferred embodiments. However, those skilled, in the art will recognize that many variations of such embodiments exist. Such variations are intended to be within the scope of the present invention and the appended claims.

Nothing in the above description is meant to limit the present invention to any specific materials, geometry, or orientation of elements. Many part/orientation substitutions are contemplated within the scope of the present invention and will be apparent to those skilled in the art. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered by way of example only to facilitate comprehension of the invention and should not be construed to limit the scope thereof.

What is claimed is:

1. A method for presentation of sequences, comprising the steps of:
extracting a first partial sequence from a mRNA;
extracting a second partial sequence from a database by searching, the second partial sequence corresponding to the first partial sequence;
predicting a first exon region within the second partial sequence using a first program;
predicting a second exon region within the second partial sequence using a second program; and
extracting a common region between the first exon region and the second exon region as a common sequence.

2. A method according to claim 1 wherein:
a plurality of the first exon regions and a plurality of the second exon regions are predicted, and common regions among the plurality of first exon regions and the plurality of second exon regions are extracted.

3. A method according to claim 1 further comprising the steps of:
predicting a third exon region within the second partial sequence using a third program; and
extracting a common region among the first, second and third exon regions as a common sequence.

4. A display system comprising:
means for displaying a first partial sequence derived from a mRNA;
means for displaying a second partial sequence derived from a genome sequence in a database, the second partial sequence corresponding to the first partial sequence;
a selection button for selecting a plurality of different programs including first and second programs;
means for displaying exon regions of the second partial sequence, the exon regions being extracted through the use of the selected plurality of different programs;
means for displaying a first exon region extracted through the use of the first program;
means for displaying a second exon region extracted through the use of the second program; and
a common sequence extraction button for extracting a sequence common to the first exon region and the second exon region.

5. A display system according to claim 4 wherein:
the common sequence extraction button is a button for extracting common sequence(s), and
the system further comprises selecting means for extracting a 5′ end sequence of any one of common sequence(s) and a 3′ end sequence of any one of common sequence(s) as a set of primers.

6. A display system according to claim 5 wherein the selecting means comprises means for selecting the length of a sequence to be amplified.

7. A display system according to claim 5 further comprising a sequence displaying means for displaying a sequence of the set of primers.

8. A display system according to claim 5 wherein the selecting means comprises means for extracting a plurality of primer sets.

9. A display system according to claim 5 further comprising sequence displaying means for displaying a sequence of a region sandwiched between two members of the selected set of primers.

10. A display system according to claim 4 further comprising a minority sequence extraction button for extracting an exon region that is predicted through the use of one of the first and second programs but is not predicted through the use of the other.

11. A method comprising the steps of:
extracting a first partial sequence from a mRNA;
identifying a second partial sequence corresponding to the first partial sequence from among a genome sequence;
identifying common sequence(s) among exon regions within the second partial sequence, the exon regions being predicted through the use of a plurality of exon prediction programs;
selecting a combination of a 5′ end sequence and a 3′ end sequence from the common sequence(s); and
designing a set of primers based on the selected combination of the 5′ end and 3′ end sequences.

12. A method according to claim 11 wherein the combination is selected based on the length of a sequence to be amplified.

13. A method according to claim 11 for cloning further comprising the steps of:
performing amplification using said set of primers; and
cloning the resulting amplified gene.


SEQUENCE LISTING (65 sequences)

SEQ ID NO: 1 - DNA, 154 bp, Homo sapiens
cacagaggaggctggcagagctggggactgagggcattgttgctgattctcactcaccggggcagcctgccgcagatgcacaggccccaggtgcaggccaccacctccgggtcggcaccaggactgccctcggtgctcatagggaatggctggg

SEQ ID NO: 2 - DNA, 154 bp, Homo sapiens
cccagccattccctatgagcaccgagggcagtcctggtgccgacccggaggtggtggcctgcacctggggcctgtgcatctgcggcaggctgccccggtgagtgagaatcagcaacaatgccctcagtccccagctctgccagcctcctctgtg

SEQ ID NO: 3 - DNA, 12437 bp, Homo sapiens
tctgtaataataagtaggaaaccaagaatgtctctgttttaagagtatcccattgcccaatttataaaacttagcaatactgcaggacttcctccaaaaaatgccgtaagaaaaatacaaagtgctgtgtttagaaattttttaaaaaaagaagcgctatggaagtggagagaagtgcaggatgaaatctgctgccctcaggctgttctcagccagggcctgagcgtggtggaggaggagtttgggtcattcctgcccagagcagggcttctctcacggagaaccttcatgctgaggtgcacatctcccagctgagactttcctagagctccatggtggtctgagactctccctacccaattcttcctcctcgtttccatgcacaagtgtcaggtctgcaccacaatctgagactctccccgcctactcttgctctttcccggtttattctttacaggcatttcccccaataaatcgcctgtatgtctgcttttcagagaccaggaactgatacaagtaaatactaagagtggtccaagaaaacagaaggccagctggggttttggaattgggtgactcacggtcctgatggcaaaaaggggctcattccaagaggaatgtgaggtataggacagaccttggtaacaagtggtggcctgaatggtgacgatttcaccagagttaacccaggagaagtgacgattatgcaggtgtgagctcacaataagagaggtggtgacagagctgaggtactagaggcaggcggtctccaggggcaggcaggatctagggtaatagaggcaggcggggtctgatagaagctgtcagaagtcattactctaatgactggcagaggtggagagacagcccacggggcctggcccttagagggggcagataggacatagtattccaaaataaataggcagctagggagggttctacataatatctgcaactaggagaaggccaggaaggaggagagggaagctggaggtggagatggagcagaagtagccccaattcctatcacttccacggggagacatccattcccaaactcccaagttctgccagggtggagatcctggtctgtctccaaaaaaggtgtctcacatgtgccagaaaaaacaaaaagaaaaaattaaataaccctaaaatgtgtcctcttcccaggagactcagcacctgcccttcgaatcccaagctaaagctgctgccagggcacttagactcttgtgtcaaggcagcagcaggtgcgaacagctgtgactttcttgacagtagtgattgctgccgatgggtaggaggaggtagagctgtctttgaacacgggaaggaacacttgtacaatccaggggtgcacttgggggcctcttggtactcccttgtcccattgtaactgttgatggacacaggcagcaattccagccttagaaatgtatgatgattatgagttcaggccccttgagtgtcaaggtttgagtcatgttacaaggtgagccatgaagacttattgaggtgatagctaagagggataggaaaatttaaaatatggtggaggaagaagaggatgaatgcccattgaagccctgagactaactgcagagagagatcctatttgtaccactgaccttctgtttctaagttccccattgagaggctcttgggaatcataaggaaactgctcccctaaatatgtacggagaaatagatttatctggtggaaggaggtagactgtggtagtgatggaagtgatctttctaaagagaagctgctctgggagcagagttgactcacagattccaacttccaccccactggagccaccatgcctctcacacttcctccaggatgttcccagacagtggctgagcacagcggggatacgagtgctggtccaagcctgcctggaccctccaatgggcaacctttgctcaggcactctccatcagcctggcaaaactacactcagacctgagctgtgatctgagactccttatatccagaccttccttattgctcttccttcacagataccaggcttttatctccatctgcgcgttctcccacaatacccctgcacctccccgtttactcttcatggaattttccctaataaatctattgcacgtctagcccatcttggtgtctgtttcttggagaactgaaactgacacactgcccaataagcctttggggagcactcctaaccctggcttatttcattgtttgcttcccagagaacatagcagcactcttgtaatcttactaaaaatgcaaatgctgggccccacacctgaagtttcttttttttttttgagacagagtctcactccattgcccaggctggagtgcagtggcatgatcttggctcactgcaacctctgcctcccaggttcaagcaattctcctgcctcagcctcccaagtagctgggactacaggcacccgtcatcatgcctggataattttagtatttttagtagagacagggtttcaccatgttggccaggctggtcttcaactcctgaccttaactgatttgcctgcctcggcctcccaaagtgctgggatttcaggtgtgagccactgtgcctggccttgaagttctttttaactaggtaagggtgggggcccaggaactggtttttcttattggtgtctcaggtacctgtgttgcagttaaaattagatgatagatactatttaattttcaacactcattactctaacactatcaaactaatataagagtactgaaattaatctttatatttaaaagaaaacaaaagaagaaattccagccaaaaggcacatgcttacccaaagggatctatggattcaatgcaatctgtgccaaaatactgatgatattcttcatagaaatggaattaaaaaccctaaaatttatatggaaccacaaaagttcacaaatagccaatacaatcttgatcaaaaagaacaaagctaaggccaggctgatgcctgtaatcccagcactttgacaggccaaggcaggtgaattgcttgagtccaggagtttgagacctgcctgggtgacatggtgaaaccccatctctacaaaaaatacaaaaattaagccgtcatggtggtgcttgcctgtagtcccagctactcaggaggctgaggtgggaggattgcttgggcatgggaggatctcttgagacagtgaggtggaggttgcagtgagccaagattgcatcactgtactccagcctgggtgacagagcaagaccatgtctcaaaaaaacaaaacaaaacaggctgggtgcagtggctcacgcctgtaatcccagcactttgggaggctgaggcagacggatcacgaagttaggagatgaagaccatcctggccaacatggtgaaaccccgtctctactaaaaatacaaaaattagctaggcgtgatggtgtgcacctgtagtcctaactactcaggaggttgaggtaggagaattgcttgaacccgggaggcagaggttgcagtgagctgagatcgtgccactgcactccagcctggtcaaaaaaaaaaaaaaaaaaaagaaatgataaatgtttgaggtgataaatatgttaattaccctaattttgttattactcattgtatgcatgtatcaaacaatcataccatacaccataaatatagataattattgtgtcaatttaaaataaaacctaaaaaggtcatgtgctgtaatattacaagaaagagcaaaacttttaagagctaaggatgaactgaaaacattgatagaatgtataaagagtggaattaatgccataaagcagggttgtcacactgtgccccttggtcaaatctggtttgctgcctgtttttatgagtttgtttgtttgtttgtttttttgagacagagtcttgctctgttgcccaggctggagtgcagtggcgcgatctcggctcactgcaagctccggctcctgggttcatgccattctcctgcctcagcctcccgagtttttatgaggttttattggaactcagtcatacacatggatcatgtattatgtatggctcagtaaacataaggaggctctgtagccagaccacttggattccaattccaattatgccactcactggttgggtgctcttgggcaggtaatttagcctctctgtgcttaagtattgttatctgcaaaatggagatagtaacaggacctacctcatagggttaatatgaggatcaaatgtgatcatgtatataccataatttcttagcacatggcctgagaggtgttaaaagagttaaaaaagtgttattttaatggatcggtttgatcatccctagaccaactcatcaaatttattatcacaaaaagaaggaaagccagacatcataggcttccagatttaatgcaattggaagtttacaatctatgaagtcttcttaccaaaaaacctggatctaattaagcatctacactatagatccaccttccagtgtacagaaaatacggggaacgaagaacatgttacatgacaccacaaggatgcaatcagcaaaatctagaatttctttaaaaaatacatcacaagtttggcacagtggtgcgtgcctgtagtctgttactcaggaggctgagacaggaggtgatatggtttggatttgtgtccccacccaaatttcatgtcgaattgtaatccccaatgttggaggaggaggggcctggtaggaggtgattggatcatgggggcagatttccctcttgctgttctcatgatggtgagttctcacgagatctgattgttcaaacgtgtgtagcacctccccttctttctcttccctgcttcccctttaccttctgccgtgattataagtttcctaaggcctcccacccatgattcctgtacagcctgcagaactgtgagccaattaaacctcttttgtttatatattacccagtctcaggtatttcttttcttttttttttttttgagacggagtctcgatctgtcgcccatgctggagtgcagtggcgcgatctcggctcactgcaagctccgccttccgggttcacgccattctcctgcctcagcctcctgagtagctgggactacaggcgcccgccaccgcgcctggctaattttttttttattttttttatttttagtagagacggggtttcaccgtgttagccaggatggtcttgatcacctgacgtcgtgatccacctgcctcggcctcgtaaagtgctgggattacaggaatgagccaccgtgcccggtccagtcccaggtatttctttatagcagtgagagaacggacctaatacaacaggattgcttgagcccaggagttcaaggctgtagtgtgggaggatagtgcttatgaatagccactaaactccagtctgggcaacatcgggagaccctttctgtaaaaaaacaaaaacaaaaacaaaaaaagggtggagactgtcagattccaagtacaggtgaggtagaagggatgtgctgtaaatataaaaaaacacaggatggaagcaaaagaatgaaaaaatccatacatgcaaacattaattgtaagaaagctgaagtggccatatttacatcagacaatgtagacattctagcaaaaatattactaaaaattttactaaatattactaaaaatttactaaattactaaaatttactaaattactaaaaatttactaaattactaaaaatttgctaaaaaatttactgcaatattactaaaaaaagataaagagggacatttagtaatgatcaaaggatcaatttatcaagaagatacggtattaatagtgtatgaatttagtaaaaaagcttcaataaacatacggaaaaatgtgacagaactgaaaaagaaaaacacaaattcagattcacagttggaaactggcctcagtctctcaggaaattatagaagaatatagaaaatcagaagggtagagaagacttgagtaacgttgtcaaacaatttgccctgactaaaatttatagacagttcacctgactgccaaatatatgttctattagtgtgtatagggaacattcagtaagatagaccatatgctgcactataatataaatcttaataaacttaaaaggattgaaatactatagagtatattatgtatagattctgtattaaattagaactcaatgacaagatgatatctggaaaatttccaaatacttggaaattagaacacacatgtctatataagccacagatccaagaagagatcataagaaatgttagaaaatgttttataccgaatgaaaatgaaatcataacattgaacttgtgggatgcagctaaagcagtgattagagggaaatttatgcctttgtccactttatcaatgcccaacctcactggcatgactaactagcaggaaagctgatgttcttcaccctggagccaaacacacacctaagactttgagaagctgaaaatagggagccagtgacagccaatggctgcttatattagaaaacgaaaaaacgtttgaaatgaggtatatgttgccttgaaaatccaaggaagcctgtttggtgccatgtttctttcttagtggctggaggggtcagatgcagccccttccctttactttgggaggtgtatcttgacatgccccagatcaatagaaatttgaatttctgtcccaccctctggcatgcatgcgtcttactgaaatgctgacagtagacactaattcctgctcatcagatccaatcagcttggtatcatcaaaacactcgatgggagtcatagcctggtgaatggagaggcagccaagatttctgcagatcagattatgacataaaatcagaaggttgataaagttctgaggtgagagagtgatggtgaattgctgccccagccagctggaagcaaactgttcctcatggtcttcactaaccacgtgtaaccacgtagccagagaacgcattcatcagaccaatagctgaataccagcaatgaaaccgtttctgaaacagcagcttcacctctgatgaagtttataataatccactgtcattctcccagaaactgcctgtcttctgcacaggcaaaattatgtaatttgaatggaaatggggtgggtgggagcagggggagtcaccatcctgcatctttcatgcttttttttttgtttttttgtttttgttttttgtttttttatttttgagacagagtctcactctgtcgccaggctggagtgtagtggcgtgatctcggttcactgcaacctctacctcctgggttcaagcgattgtcctgcctcagcctcccgagtagctgggactacaggcttgcggcaccacgcccagctaatttttgtagttttagtagagacggggttttaccatgttggcaaggatggtcttgatctcttgacctcgtgatccgtctgcctcagcctcccaaagtgctgggattacaggcacggtggttcatgttcttgatggtagcactaaactctgcaattcctccagcaatgtgaaatgcctttttcctatgttggtagatagaggcagtttcagtgccttctgcttggctcagagaatccatgtagggactctgccagtttctagtagatatattcaaatgatgcattctggaactgaagaaataactacaggattcaagaacccactggcttatctgagacgaacctatgcccaaactcgactgatcattttttctatcaactagtattctagctgatagacatagtggtattttagctctccaggaattagtgtcagttcagagccattgacccgcaattgctgagtagtcgttatttccctttcttcagtgcatagtcaatatacaacctagtaaatatccatgagattctttgggaaggtcaggaggacgagttacagtacaaactctgtggctcagagttcaggtgtacctggacccactttcattcaaggagctctgggtctgtgaactggctcaactctaggaattggttgggaggctgaaactccctattaaaatgatttttctttactgctcttcgccagacctagagctttcctgcttgtaaaaatcaagtaaggcttcagtaggctgccatgtgtttcagctctagggatgctgtcactgactaatcattgcccaagatctctgaggacagaccatctggatgactgtgccagttctgctgcccatgatggcagctggtcccacactggctttggcaattaagtgccaccactcagtcatcagattctatcattcccagtgaattcagcgagtaaagcttaatgacagcaacttccccaccatcattaccgacctagactccccaaggattaagaagcccccacccccgacccccaccgtcctgacaaatggacttctcagtcttgtgagggggttttcttcagattctgagagccagagttaggcgatgggtggacaggttttacatgaaaaatctactccagcattctaagcctttaggagacatttttccaaagtgtactgagaaagttctggcatttcacctgtagaaacatgcctagccatttgaacaagcaaactgactttagaattgtgatgtggtaaacctgattaaaaattttggcctgatccaacattattttccttccaatctaatccagtatccctttgatccattcctatataagttctacagatttctgccaatgtcacttggcaaaaatcttgcaacttttatggtgcgtgttgtgccttattaggcctcaaacccctctgaaccttacttacctttcagggccgggcaggccgagtctgggttacaggtctagaagcagtgacaggtgaggtggggggtagatcctgaggaggatccccagctctttgcaaatcagcttccactggtgaggccatcatagattcatcaggcaagattaaccctctgagtaagcacaggtggattccttctactggccaaagaaggctcagcagaattcaggggtttaaggtcttcagcttcatgaaaatctgcccatttgctcccattccattcttcagcatcccactcctttccaattagttctctacctttaccatgagagactctgcagagttgagaatagaatctgcattgtttcagtcacctgcaagcttatactttgagtttagctttcagaaatcttggctgtgtggctacgagagataagggtttcttgcagggtggccatataagatttctggtccctggcctggtctttgagctggagaaaaccctaaactgatgactttgttttccttaaatccaatcagatcaggatttcttgtgccagccaagatggtgacagcccattacagtctctgtctgccacagctacaaataaaaaccagacaagctaccaaaaacaacttcctaagggctttgtttttttttttttaattttttttagagatgggggactcactttgttgtccaggctggtctcaaactcctgggtttaagcgattttcacaagtagctgggattacaggtgtgtgccactgcacccagcaacttcctaaggactcggaaaagttaacaatggcaggaagattggagagaggaaccaaaacttgaagaaccctatatatttttcaatactcttatatttaggaatagaatcagtcaatataaattgtaaatgaaatgcttcttcacagcctttggaggatgggttataccagtgattgccaaacttgcagcaatagttaaaaaaaattatagggctgcctccccgtctctccctggagcccagaatctccaggagaaacccagaaatatacatgtttaacaaaaccttgatttctcgagttagtaaatttaggaaaaatggcttatccaagcaaatagaattggatttcctaaaaatttagtcctcacccaagctatttattttggaggcaaaagaagagtatcttatgacctagaagactctaaaaatgtggtatgaagactgtctttattagaaaaaccaggggttaacacatctcatttaacaatgtgtagggggtccccaactacaccactttccaaatagcggtcttatcagattttaaaatgctgatacaaaagttatgatcttataaaatgacatttacttccaacatggtgaaaccccatctttactaaaaatattttaaaaaccagctgggtgtggtggtgggcacctgtaatcccagctactctggaggctgaggccagagaattgcttgaacccaggagacggagatggcagtgagacaacacggtgccactgcactctagcctcggggacagagtgagactccgtctcaaaaaacaacaacaacaaaaaaccacatgtggtagttagtttaaaaatcacagaccttaggctaggtaatcagcaaatatttgctaaatgagtaatagaaactttgtcaatatcatagaggttcttacaatagattcaaaaggtaattggaaccaacatgcaataaaaagagattaacacaaaaatagataagtggattactatttaatgaatatttttggaaaaactctatttcagtttagaaaagaatgaactgagctcctatattttcattctacacattgcagtgaattccagatgggactcaaagacttagcaacaacaacagaacagaacagaataatttatattttctggctcttgctggggaaatacattctaaatattgaagaaaaaggaaatactttcagggaatgcatttctgaatatatatgtaaactttgcaaagctttttctgtaatgaaatcttttaatattttggtcaagcaacagaatggaggaaaaacaaataataagcaaatacttgcttattaatatgtttaggaaacacttggataacacacatacacatatgaatatatacacacacacatatatatatatgttctacatgatcatactaaaatttagatgacacttcgtccaggaagaaatattgaagaaaatgcaatcatacaaacacgacattgccacaggggaaaacataacatccatggtaaatataacgatacaaaaatctgttacatttagtaaaaagtgcagaaaaccggcaagaaaaatgcaggagcctgattttggtagaaagtggcaaaaaggctctaaatgcccagtgcaaatggcttgaaattccattcccaggcctggggctccacatttccaggggctctgctttccaaaagatatggttcaatgtcccctaggtatagtgccttcggtctggactgcagatggagacctgggtcatcccagaagctttgtagtgtgagagatcaaactggctgctctgccaggcctcacctatttccagactggatgattcctgatacttttacttttgagcccaaactaacttctgcatggaggttcagtacaggtcaggtgagtgacgaatctcctgacagggctacagtgaactttcacaaggttaaaccttctaaacttgggaagctgctttgcaaaaataaataaataaaataaaaatcccttactcaattgttgactgggtattattccggaaacaggaaacaggtttttcatttctgccaggttgtgccccagcttccatttgctaagcagcttaggggaacattggattaaaaaccacccgtaaagaaaaggcattaagaaattgatttgaacgatttgaaattattatcttgaaagatcaaagaaacagtgattatgaacaccggctctggtgggaagcggttcttgaaagttaaagcatgggggaactcgagtactctggagcatcatcagcatctgtacaatgcaagtgaactctggaagctagacgcatgctttgcacgtggacacagacacatgcttgcacaggacacatttgcaatacatacatgtttatacatggacacatgttttgcacacgcttgcacaggacacgcttgcacaggacacatgcacaggatgcatgttttgtacaggacacatgttttgcacatggaaacacatatccatgctttgcaaagggacacagggaagcatgcttacaatggatacaagtttacacacggacgcatgttttgcacaggacatatgcacaggatgcatgttttgcacacggacacacacgtttacataggacaagtgcacaggacacgttttgcaccggacgtattctgtgcacgggacgcacggtttgcagatgcaagcacacacactcctcggcgcgacgggcgaagcggggtgggcagcgacagcggagtccggacccagggacagcgcctccgggaacctcgccaaggcggcgggaactacaactcccggctcggtcgcctcgggacgacgcgcggcccagcgcagactggccccgcccagcggccccgcgcggcccggcccaggtcccggcgcccagagtcgccgcgcggccgccg

SEQ ID NO: 4 - DNA, 133 bp, Homo sapiens
ctttctgggaccctggggggctatgtcactcaggactcttgacttttccctgagagctgaggatgagcccattgagagcagcatcagaaaaccctccctgggggcttccggtgcctgccctggttcttccagg

SEQ ID NO: 5 - DNA, 175 bp, Homo sapiens
ctgctggtggtctccggccagcagaacctggctgtgcagcagccagctggcgagatggcccggcacactgctgggtggcttgcaaggcagggagcgatggaggggctctggtctcagagggcccgggaactgagcagaaaaattgggcagagactcaatcagtcacccgacttag

SEQ ID NO: 6 - DNA, 119 bp, Homo sapiens
agtttggtgtcactggacacgactgtgtgcatgacggggagcctggggacagggcctgtgactgcatggtcgtggaccttgttccttgcctgggttccaccctggaggagggagccctt

SEQ ID NO: 7 - DNA, 184 bp, Homo sapiens
atttggctgcttactgctcaaaagccaaatacgagagacaagagttggtggcaggtttatctggagagccagcaaaccaggaagatggtagataagtgtctaaagtgccgtctaagtcagtacaggtttcgggttcttaattatgttaagggcagtgggaaaaagaggctgttgggatcaagag

SEQ ID NO: 8 - DNA, 188 bp, Homo sapiens
gtttctgggtggagcggacccctgtgcacgaggcagcccagcggggtgagagcctgcagctgcaacagctgatcgagagcggcgcctgcgtgaaccaggtcaccgtggactccatcacgcccctgcacgcagccagtctgcagggccaggcgcggtgtgtgcagctgctgctggcggctggggcccag

SEQ ID NO: 9 - DNA, 123 bp, Homo sapiens
cacataatagcaaagcaaaccggtgcttctaagaagcatcttagcaaagatgaacctgcagcaggatgggaaagtgggttgcgtggcatttctctgccctttggacacttgtttcttcctccc

SEQ ID NO: 10 - DNA, 151 bp, Homo sapiens
gtggatgctcgcaacatcgacggcagcaccccgctctgcgatgcctgcgcctcgggcagcatcgagtgtgtgaagctcttgctgtcctacggggccaaggtcaaccctcccctgtacacagcgtcccccctgcacgaggcctgcatgagcg

SEQ ID NO: 11 - DNA, 135 bp, Homo sapiens
ggagttccgaatgtgtgaggcttcttattgacgtcggggccaatctggaagcgcacgattgccattttgggacccctctgcacgttgcctgtgcccgggagcatctggactgtgtcaaagtgctgctcaatgcag

SEQ ID NO: 12 - DNA, 351 bp, Homo sapiens
cgggtgtttacctgcgtgggtgtttgcccaagtgggtgtttgcccgagtgggtgtttacccgagtgggtgtttaccggagtggatgtttgcccgcgtgggtgtttaccggagtgggtgtttgcccgcgtgggtgtttaccccagtgggtgtttgcctgagtgggtgtttgcccgtgtgggtgtttacctgcgtgggtgtttacccccgggggtgtttgcccgcgtgggtgtttacccgagggggtgtttgcccgagtgggtgtttgcccgcggcggtgtttaccagagtggttgtttaccccagtgggtgtttacccgagggggtgtttgcctgcgtgggtgtttgcctgc

SEQ ID NO: 13 - DNA, 192 bp, Homo sapiens
gggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaa

SEQ ID NO: 14 - DNA, 47 bp, Homo sapiens
gtggggtccggaccgtggctgcccccgttgtgcccagcattgcccgg

SEQ ID NO: 15 - DNA, 607 bp, Homo sapiens
atagaacaacgctccttcgagtcccttcctgcgatcctgtttaggcttctctcctggatcctggataatgtttccagggtgttgggaaggcctgcgtctcaggtcacagttgtgggtgtggccctgcgctgttctacagaacctaccctctcaatgggcatgggcccaaccatccagttttcctcttttacggaccatcctcaaaggcactctcaggacagacggcgtggggagcacagaggaggctggcagagctggggactgagggcattgttgctgattctcactcaccggggcagcctgccgcagatgcacaggccccaggtgcaggccaccacctccgggtcggcaccaggactgccctcggtgctcatagggaatggctgggcccacggaaggtcggcctgggatgtggcctgggactgctgctctgctggctgctgtgtggatgcttttcctggagcactttccaaggcatcccccagccccaagcctgcgcgcatctgtcactcagggactttctatgggtctttgtgggggaaggccctggctttgtattccccacaagtagcactgagtttcttaggaaatttgtcttcagtattag

SEQ ID NO: 16 - DNA, 129 bp, Homo sapiens
acagtgtggaacagagtgagcctgagctgcccaaggcgatgccctttgtgctgtatgactggaggcctctccgcgtgggtacttgggggtggggccacaaatctggcgaaaaacttcatgctggccgcc

SEQ ID NO: 17 - DNA, 197 bp, Homo sapiens
aaacaccaagatgggcgcatcctgtgtggatggagaggggtggctctcgtcttgctgtgtcccaggtcgtttggcagcgctgtgcttgccagggagccacactttcccatctcttccccaggtctcctgatgccaacttcagcaccccccttatgtggacttttcttgggggagacagacgctgtccacgggaccag

SEQ ID NO: 18 - DNA, 636 bp, Homo sapiens
gtgggaggcgtcgtgagggagacagcaagagaccccagtgggatccccagtgggatctcttgctcagctctgggctccccgtggtcttccagggagggagtggaatctcgtctactcctggaccatagcaatgctccctgtggtatcccatctgtgctaccaggtgagtctgcagagagctctgaagtcatttgggatgaaaagaatgcctttccctgtccatttcatgccctgggccactttgttaaactgcttgtgctttctctccagtgggcaacacctgtttcttctaggcagttgagttcctttattcggaaaaacgagcgtaagactggctctgagaacaaagctggacagcttgctcatctttcgagcagctgtgccgtggagagatgaaggtggggcgcacagggccctggcaggggctgtgccccctgtaatggctgagaaaatatttccagaccctggagtctttgccttttctcttttctccacattagcaccacatgacagtgacagcgaggcctcagtgattttggcttgaaggtcttgtgatctccgacaagttgaatgaaaagatgtcttaaattggtccaatctaaagagtgcctcctttttctccaaaccatgaaaaaa

SEQ ID NO: 19 - DNA, 2071 bp, Homo sapiens
gtttaaaaatcacagaccttaggctaggtaatcagcaaatatttgctaaatgagtaatagaaactttgtcaatatcatagaggttcttacaatagattcaaaaggtaattggaaccaacatgcaataaaaagagattaacacaaaaatagataagtggattactatttaatgaatatttttggaaaaactctatttcagtttagaaaagaatgaactgagctcctatattttcattctacacattgcagtgaattccagatgggactcaaagacttagcaacaacaacagaacagaacagaataatttatattttctggctcttgctggggaaatacattctaaatattgaagaaaaaggaaatactttcagggaatgcatttctgaatatatatgtaaactttgcaaagctttttctgtaatgaaatcttttaatattttggtcaagcaacagaatggaggaaaaacaaataataagcaaatacttgcttattaatatgtttaggaaacacttggataacacacatacacatatgaatatatacacacacacatatatatatatgttctacatgatcatactaaaatttagatgacacttcgtccaggaagaaatattgaagaaaatgcaatcatacaaacacgacattgccacaggggaaaacataacatccatggtaaatataacgatacaaaaatctgttacatttagtaaaaagtgcagaaaaccggcaagaaaaatgcaggagcctgattttggtagaaagtggcaaaaaggctctaaatgcccagtgcaaatggcttgaaattccattcccaggcctggggctccacatttccaggggctctgctttccaaaagatatggttcaatgtcccctaggtatagtgccttcggtctggactgcagatggagacctgggtcatcccagaagctttgtagtgtgagagatcaaactggctgctctgccaggcctcacctatttccagactggatgattcctgatacttttacttttgagcccaaactaacttctgcatggaggttcagtacaggtcaggtgagtgacgaatctcctgacagggctacagtgaactttcacaaggttaaaccttctaaacttgggaagctgctttgcaaaaataaataaataaaataaaaatcccttactcaattgttgactgggtattattccggaaacaggaaacaggtttttcatttctgccaggttgtgccccagcttccatttgctaagcagcttaggggaacattggattaaaaaccacccgtaaagaaaaggcattaagaaattgatttgaacgatttgaaattattatcttgaaagatcaaagaaacagtgattatgaacaccggctctggtgggaagcggttcttgaaagttaaagcatgggggaactcgagtactctggagcatcatcagcatctgtacaatgcaagtgaactctggaagctagacgcatgctttgcacgtggacacagacacatgcttgcacaggacacatttgcaatacatacatgtttatacatggacacatgttttgcacacgcttgcacaggacacgcttgcacaggacacatgcacaggatgcatgttttgtacaggacacatgttttgcacatggaaacacatatccatgctttgcaaagggacacagggaagcatgcttacaatggatacaagtttacacacggacgcatgttttgcacaggacatatgcacaggatgcatgttttgcacacggacacacacgtttacataggacaagtgcacaggacacgttttgcaccggacgtattctgtgcacgggacgcacggtttgcagatgcaagcacacacactcctcggcgcgacgggcgaagcggggtgggcagcgacagcggagtccggacccagggacagcgcctccgggaacctcgccaaggcggcgggaactacaactcccggctcggtcgcctcgggacgacgcgcggcccagcgcagactggccccgcccagcggccccgcgcggcccggcccaggtcccggcgcccagagtcgccgcgcggccgc

SEQ ID NO: 20 - DNA, 87 bp, Homo sapiens
catctttgcgtgtggcagcggaagacgctaaatccacctggacgtcttgtgaatggaggcacgtcgggaacgcgccttcccggctca

SEQ ID NO: 21 - DNA, 186 bp, Homo sapiens
ttctgggtggagcggacccctgtgcacgaggcagcccagcggggtgagagcctgcagctgcaacagctgatcgagagcggcgcctgcgtgaaccaggtcaccgtggactccatcacgcccctgcacgcagccagtctgcagggccaggcgcggtgtgtgcagctgctgctggcggctggggcccag

SEQ ID NO: 22 - DNA, 150 bp, Homo sapiens
gtggatgctcgcaacatcgacggcagcaccccgctctgcgatgcctgcgcctcgggcagcatcgagtgtgtgaagctcttgctgtcctacggggccaaggtcaaccctcccctgtacacagcgtcccccctgcacgaggcctgcatgagc

SEQ ID NO: 23 - DNA, 132 bp, Homo sapiens
agttccgaatgtgtgaggcttcttattgacgtcggggccaatctggaagcgcacgattgccattttgggacccctctgcacgttgcctgtgcccgggagcatctggactgtgtcaaagtgctgctcaatgca

SEQ ID NO: 24 - DNA, 189 bp, Homo sapiens
gccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaa

SEQ ID NO: 25 - DNA, 45 bp, Homo sapiens
ggggtccggaccgtggctgcccccgttgtgcccagcattgcccgg

SEQ ID NO: 26 - DNA, 294 bp, Homo sapiens
gtcacagttgtgggtgtggccctgcgctgttctacagaacctaccctctcaatgggcatgggcccaaccatccagttttcctcttttacggaccatcctcaaaggcactctcaggacagacggcgtggggagcacagaggaggctggcagagctggggactgagggcattgttgctgattctcactcaccggggcagcctgccgcagatgcacaggccccaggtgcaggccaccacctccgggtcggcaccaggactgccctcggtgctcatagggaatggctgggcccacgga

SEQ ID NO: 27 - DNA, 75 bp, Homo sapiens
tctcctgatgccaacttcagcaccccccttatgtggacttttcttgggggagacagacgctgtccacgggaccag

SEQ ID NO: 28 - DNA, 366 bp, Homo sapiens
tgggcaacacctgtttcttctaggcagttgagttcctttattcggaaaaacgagcgtaagactggctctgagaacaaagctggacagcttgctcatctttcgagcagctgtgccgtggagagatgaaggtggggcgcacagggccctggcaggggctgtgccccctgtaatggctgagaaaatatttccagaccctggagtctttgccttttctcttttctccacattagcaccacatgacagtgacagcgaggcctcagtgattttggcttgaaggtcttgtgatctccgacaagttgaatgaaaagatgtcttaaattggtccaatctaaagagtgcctcctttttctccaaaccatgaaaaaa

SEQ ID NO: 29 - DNA, 2071 bp, Homo sapiens; identical to SEQ ID NO: 19

SEQ ID NO: 30 - DNA, 186 bp, Homo sapiens; identical to SEQ ID NO: 21

SEQ ID NO: 31 - DNA, 150 bp, Homo sapiens; identical to SEQ ID NO: 22

SEQ ID NO: 32 - DNA, 132 bp, Homo sapiens; identical to SEQ ID NO: 23

SEQ ID NO: 33 - DNA, 189 bp, Homo sapiens; identical to SEQ ID NO: 24

SEQ ID NO: 34 - DNA, 45 bp, Homo sapiens; identical to SEQ ID NO: 25

SEQ ID NO: 35 - DNA, 294 bp, Homo sapiens; identical to SEQ ID NO: 26

SEQ ID NO: 36 - DNA, 75 bp, Homo sapiens; identical to SEQ ID NO: 27

SEQ ID NO: 37 - DNA, 366 bp, Homo sapiens; identical to SEQ ID NO: 28

SEQ ID NO: 38 - DNA, 22 bp, Artificial sequence
  partial sequence of AL365356
gaaacagtgattatgaacaccg

SEQ ID NO: 39 - DNA, 17 bp, Artificial sequence
  partial sequence of AL365356
gcgaccgagccgggagt

SEQ ID NO: 40 - DNA, 17 bp, Artificial sequence
  partial sequence of AL365356
ggagcggacccctgtgc

SEQ ID NO: 41 - DNA, 16 bp, Artificial sequence
  partial sequence of AL365356
cagccgccagcagcag

SEQ ID NO: 42 - DNA, 17 bp, Artificial sequence
  partial sequence of AL365356
cgcaacatcgacggcag

SEQ ID NO: 43 - DNA, 18 bp, Artificial sequence
  partial sequence of AL365356
caggggggacgctgtgta

SEQ ID NO: 44 - DNA, 22 bp, Artificial sequence
  partial sequence of AL365356
tgtgtgaggcttcttattgacg

SEQ ID NO: 45 - DNA, 22 bp, Artificial sequence
  partial sequence of AL365356
gcagcactttgacacagtccag

SEQ ID NO: 46 - DNA, 19 bp, Artificial sequence
  partial sequence of AL365356
gagactgcccttcaccacg

SEQ ID NO: 47 - DNA, 16 bp, Artificial sequence
  partial sequence of AL365356
agcacttggcgggagc

SEQ ID NO: 48 - DNA, 15 bp, Artificial sequence
  partial sequence of AL365356
ccggaccgtggctgc

SEQ ID NO: 49 - DNA, 16 bp, Artificial sequence
  partial sequence of AL365356
gggcaatgctgggcac

SEQ ID NO: 50 - DNA, 22 bp, Artificial sequence
  partial sequence of AL365356
tacagaacctaccctctcaatg

SEQ ID NO: 51 - DNA, 17 bp, Artificial sequence
  partial sequence of AL365356
ctgcacctggggcctgt

SEQ ID NO: 52 - DNA, 19 bp, Artificial sequence
  partial sequence of AL365356
tgatgccaacttcagcacc

SEQ ID NO: 53 - DNA, 17 bp, Artificial sequence
  partial sequence of AL365356
cccgtggacagcgtctg

SEQ ID NO: 54 - DNA, 23 bp, Artificial sequence
  partial sequence of AL365356
gtttcttctaggcagttgagttc

SEQ ID NO: 55 - DNA, 22 bp, Artificial sequence
  partial sequence of AL365356
ccttcaagccaaaatcactgag

SEQ ID NO: 56 - DNA, 22 bp, Artificial sequence
  misc_feature (22): partial sequence of AL365356 , n is a, c, g or t
ttttttttttttttttttttvn

SEQ ID NO: 57 - DNA, 413 bp, Artificial sequence
  partial sequence of AL365356
gaggcagcccagcggggtgagagcctgcagctgcaacagctgatcgagagcggcgcctgcgtgaaccaggtcaccgtggactccatcacgcccctgcacgcagccagtctgcagggccaggcgcggtgtgtgcagctgctgctggcggctggggcccaggtggatgctcgcaacatcgacggcagcaccccgctctgcgatgcctgcgcctcgggcagcatcgagtgtgtgaagctcttgctgtcctacggggccaaggtcaaccctcccctgtacacagcgtcccccctgcacgaggcctgcatgagcgggagttccgaatgtgtgaggcttcttattgacgtcggggccaatctggaagcgcacgattgccattttgggacccctctgcacgttgcctgtgcccgggagca

SEQ ID NO: 58 - DNA, 549 bp, Artificial sequence
  partial sequence of AL365356
cttcttcccgcggttgtcccgggcgtagatgttgccgccaaactcgataagcatctcgatgaggtcaacattcttgaccttggccgcgtggtgaagggcagtctcatgaagctttgccgcattcacgttggcccctgcattgagcagcactttgacacagtccagatgctcccgggcacaggcaacgtgcagaggggtcccaaaatggcaatcgtgcgcttccagattggccccgacgtcaataagaagcctcacacattcggaactcccgctcatgcaggcctcgtgcaggggggacgctgtgtacaggggagggttgaccttggccccgtaggacagcaagagcttcacacactcgatgctgcccgaggcgcaggcatcgcagagcggggtgctgccgtcgatgttgcgagcatccacctgggccccagccgccagcagcagctgcacacaccgcgcctggccctgcagactggctgcgtgcaggggcgtgatggagtccacggtgacctggttcacgcaggcgccgctctcgatcagctgttgcag

SEQ ID NO: 59 - DNA, 616 bp, Artificial sequence
  partial sequence of AL365356
tgcaacagctgatcgagagcggcgcctgcgtgaaccaggtcaccgtggactccatcacgcccctgcacgcagccagtctgcagggccaggcgcggtgtgtgcagctgctgctggcggctggggcccaggtggatgctcgcaacatcgacggcagcaccccgctctgcgatgcctgcgcctcgggcagcatcgagtgtgtgaagctcttgctgtcctacggggccaaggtcaaccctcccctgtacacagcgtcccccctgcacgaggcctgcatgagcgggagttccgaatgtgtgaggcttcttattgacgtcggggccaatctggaagcgcacgattgccattttgggacccctctgcacgttgcctgtgcccgggagcatctggactgtgtcaaagtgctgctcaatgcaggggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaaagacacctct

SEQ ID NO: 60 - DNA, 1118 bp, Artificial sequence
  partial sequence of AL365356
caacagctgatcgagagcggcgcctgcgtgaaccaggtcaccgtggactccatcacgcccctgcacgcagccagtctgcagggccaggcgcggtgtgtgcagctgctgctggcggctggggcccaggtggatgctcgcaacatcgacggcagcaccccgctctgcgatgcctgcgcctcgggcagcatcgagtgtgtgaagctcttgctgtcctacggggccaaggtcaaccctcccctgtacacagcgtcccccctgcacgaggcctgcatgagcgggagttccgaatgtgtgaggcttcttattgacgtcggggccaatctggaagcgcacgattgccattttgggacccctctgcacgttgcctgtgcccgggagcatctggactgtgtcaaagtgctgctcaatgcaggggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaaagacacctctgactctgtcacagctctgcagggtgaacttgaggaaggccactggcgtccgagggctggagaagattgccaagttaaacatcccgccccggctcattgattacctctcctacaactgaattgcaggtggggtccggaccgtgactgcccccgttgtgcccagcattgcccgggtgagggctctgcctgttcctctgaagcagcgtgattgctgtagatagaacaacgctccttcgagtcccttcctgcgatcctgtttaggcttctctcctggatcctggataatgtttccagggtgttgggaaggcctgcgtctcaggtcacagttgtgggtgtggccctgcgctgttctacagaacctaccctctcaatgggcatgggcccaaccatccagttttcctcttttacggaccatcctcaaaggcactctcaggacagacggcgtggggagcacagaggaggctggcagagctggggactcagggcattgttgctgattctcact

SEQ ID NO: 61 - DNA, 550 bp, Artificial sequence
  partial sequence of AL365356
catcgagtgtgtgaagctcttgctgtcctacggggccaaggtcaaccctcccctgtacacagcgtcccccctgcacgaggcctgcatgagcgggagttccgaatgtgtgaggcttcttattgacgtcggggccaatctggaagcgcacgattgccattttgggacccctctgcacgttgcctgtgcccgggagcatctggactgtgtcaaagtgctgctcaatgcaggggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaaagacacctctgactctgtcacagctctgcagggtgaacttgaggaaggccactggcgtccgagggctggagaagattgccaagttaaacatcccgccccggctcattgattacctctcctacaactgaatt

SEQ ID NO: 62 - DNA, 926 bp, Artificial sequence
  misc_feature (909): partial sequence of AL365356 , n is a, c, g or t
catcgagtgtgtgaagctcttgctgtcctacggggccaaggtcaaccctcccctgtacacagcgtcccccctgcacgaggcctgcatgagcgggagttccgaatgtgtgaggcttcttattgacgtcggggccaatctggaagcgcacgattgccattttgggacccctctgcacgttgcctgtgcccgggagcatctggactgtgtcaaagtgctgctcaatgcaggggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaaagacacctctgactctgtcacagctctgcagggtgaacttgaggaaggccactggcgtccgagggctggagaagattgccaagttaaacatcccgccccggctcattgattacctctcctacaactgaattgcaggtggggtccggaccgtgactgcccccgttgtgcccagcattgcccgggtgagggctctgcctgttcctctgaagcagcgtgattgctgtagatagaacaacgctccttcgagtcccttcctgcgatcctgtttaggcttctctcctggatcctggataatgtttccagggtgttgggaaggcctgcgtctcaggtcacagttgtgggtgtggccctgcgctgttctacagaacctaccctctcaatgggcatgggcccaaccatccagttttcctcttttacggaccatcctcaaaggcactctcaggacagacggcgtggggagcacagaggaggctggcagagctggggactnagggcattgttgctgat

SEQ ID NO: 63 - DNA, 796 bp, Artificial sequence
  misc_feature (188): partial sequence of AL365356 , n is a, c, g or t
tgcggcaggctgccccggtgagtgagaatcagcaacaatgccctgagtccccagctctgccagcctcctctgtgctccccacgccgtctgtcctgagagtgcctttgaggatggtccgtaaaagaggaaaactggatggttgggcccatgcccattgagagggtaggttctgtagaacagcgcagggncacacccacaactgtgacctgagacgcaggccttcccaacaccctggaaacattatccaggatccaggagagaagcctaaacaggatcgcaggaagggactcgaaggagcgttgttctatctacagcaatcacgctgcttcagaggaacaggcagagccctcacccgggcaatgctgggcacaacgggggcagtcacggtccggaccccacctgcaattcagttgtaggagaggtaatcaatgagccggggcgggatgtttaacttggcaatcttctccagccctcggacgccagtggccttcctcaagttcaccctgcagagctgtgacagagtcagaggtgtcttttcgtagtactcgaagcacttggcgggagcgctgctgctccacgtgtagtcagacggcttcttcccgcggttgtcccgggcgtagatgttgccgccaaactcgataagcatctcgatgaggtcaacattcttgaccttggccgcgtggtgaagggcagtctcatgaagctttgccgcattcacgttggcccctgcattgagcagcactttgacacagtccagatgctcccgggcacaggcaacgtgcagaggggtcccaaa

SEQ ID NO: 64 - DNA, 1251 bp, Artificial sequence
  partial sequence of AL365356
gaggcagcccagcggggtgagagcctgcagctgcaacagctgatcgagagcggcgcctgcgtgaaccaggtcaccgtggactccatcacgcccctgcacgcagccagtctgcagggccaggcgcggtgtgtgcagctgctgctggcggctggggcccaggtggatgctcgcaacatcgacggcagcaccccgctctgcgatgcctgcgcctcgggcagcatcgagtgtgtgaagctcttgctgtcctacggggccaaggtcaaccctcccctgtacacagcgtcccccctgcacgaggcctgcatgagcgggagttccgaatgtgtgaggcttcttattgacgtcggggccaatctggaagcgcacgattgccattttgggacccctctgcacgttgcctgtgcccgggagcatctggactgtgtcaaagtgctgctcaatgcaggggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaaagacacctctgactctgtcacagctctgcagggtgaacttgaggaaggccactggcgtccgagggctggagaagattgccaagttaaacatcccgccccggctcattgattacctctcctacaactgaattgcaggtggggtccggaccgtgactgcccccgttgtgcccagcattgcccgggtgagggctctgcctgttcctctgaagcagcgtgattgctgtagatagaacaacgctccttcgagtcccttcctgcgatcctgtttaggcttctctcctggatcctggataatgtttccagggtgttgggaaggcctgcgtctcaggtcacagttgtgggtgtggccctgcgctgttctacagaacctaccctctcaatgggcatgggcccaaccatccagttttcctcttttacggaccatcctcaaaggcactctcaggacagacggcgtggggagcacagaggaggctggcagagctggggactcagggcattgttgctgattctcactcaccggggcagcctgccgcagatgcacaggccccaggtgcaggccaccacctccgggtcggcaccaggactgccctcggtgctcatagggaatggctggg

SEQ ID NO: 65 - DNA, 462 bp, Artificial sequence
  partial sequence of AL365356
atgagcgggagttccgaatgtgtgaggcttcttattgacgtcggggccaatctggaagcgcacgattgccattttgggacccctctgcacgttgcctgtgcccgggagcatctggactgtgtcaaagtgctgctcaatgcaggggccaacgtgaatgcggcaaagcttcatgagactgcccttcaccacgcggccaaggtcaagaatgttgacctcatcgagatgcttatcgagtttggcggcaacatctacgcccgggacaaccgcgggaagaagccgtctgactacacgtggagcagcagcgctcccgccaagtgcttcgagtactacgaaaagacacctctgactctgtcacagctctgcagggtgaacttgaggaaggccactggcgtccgagggctggagaagattgccaagttaaacatcccgccccggctcattgattacctctcctacaactga